(19) United States
(12) Patent Application Publication
(10) Pub. No.: US 2026/0091051 A1
(43) Pub. Date: Apr. 2, 2026

(54) USE OF NMN FOR THE PREVENTION AND/OR TREATMENT OF PAIN, AND CORRESPONDING COMPOSITIONS

(71) Applicant: Nuvamid SA, Epalinges (CH)

(72) Inventors: Guillaume BERMOND, Epalinges (CH); Laurent GARÇON, Epalinges (CH)

(21) Appl. No.: 19/345,274

(22) Filed: Sep. 30, 2025

Related U.S. Application Data
(62) Division of application No. 17/640,204, now abandoned.

(30) Foreign Application Priority Data
Sep. 9, 2019 (FR) 1909896
Dec. 20, 2019 (EP) 19218817.5

(51) Int. Cl. A61K 31/7084; A61K 45/06; A61P 29/00

(52) U.S. Cl. CPC A61K 31/7084; A61K 45/06; A61P 29/00

(57) ABSTRACT

A method for preventing and/or treating pain, in particular visceral nociceptive pain, includes administering to a subject nicotinamide mononucleotide, a pharmaceutically acceptable precursor thereof, a pharmaceutically acceptable derivative thereof, or a pharmaceutically acceptable salt thereof.

### CROSS-REFERENCES TO RELATED APPLICATIONS

This application is a divisional of U.S. application Ser. No. 17/640,204 filed Mar. 3, 2022, which is a § 371 national stage entry of International Application No. PCT/EP2020/075085, filed Sep. 8, 2020, which claims priority to French Patent Application No. FR1909896, filed Sep. 9, 2019, and European Patent Application No. 19218817.5, filed Dec. 20, 2019, the entire contents of which are incorporated herein by reference.

### FIELD

The present invention relates to the use of nicotinamide mononucleotide (NMN), a pharmaceutically acceptable derivative thereof, or a pharmaceutically acceptable salt thereof, for the treatment and/or prevention of pain, in particular nociceptive pain. The present invention also relates to compositions that comprise NMN, a pharmaceutically acceptable derivative thereof, or a pharmaceutically acceptable salt thereof for the treatment and/or prevention of pain, in particular nociceptive pain.

### BACKGROUND

Pain can be both a symptom and a disease. Pain is defined by the International Association for the Study of Pain (IASP) as “An unpleasant sensory and emotional experience associated with, or resembling that associated with, actual or potential tissue damage.” (see the definition at the following link according to the updated version dated 14 Dec. 2017: www.iasp-pain.org/resources/terminology/#pain). Pain is information that is processed by the nervous system.

The nervous system of mammals like humans has two main parts:
the central nervous system is constituted of the brain (encephalon), the brainstem, the cerebellum and the spinal cord. The role of the central nervous system is to receive, register, and interpret the signals that come from the peripheral nervous system, and
the peripheral nervous system is constituted of the cranial and spinal nerves, attached to the central nervous system, and corresponding endings thereof. The role of the peripheral nervous system is to (i) transmit the information received by the peripheral sensitivity and pain receptors to the central nervous system and (ii) transmit the orders sent by the central nervous system in particular to the muscles.

Pain is a complex phenomenon and consequently it can thus be categorised in different ways. There are three main categories of pain: nociceptive pain, neuropathic pain, and centralised pain (also known as central sensitisation).

Nociceptive or peripheral pain is caused by the activation of nociceptors. Nociceptors are receptors situated at the end of nerve fibers. In the case of nociceptive pain, the nervous system is not affected. Nociceptive pain may be induced by various different stimuli such as a mechanical stimulus, a thermal stimulus, a chemical stimulus, an inflammatory disease or an infectious disease. An example of nociceptive pain that may be cited is pain due to burning of the skin.

Neuropathic pain is caused by lesions to the nerves of the peripheral nervous system. An example that may be cited is diabetic neuropathic pain.

Centralised pain is due to a disruption in processing of pain by the central nervous system. Examples that may be cited include fibromyalgia or phantom limb pain.

In contrast, migraine is a category of pain that is distinct from these aforementioned three categories and thus, for the time being, constitutes a separate category.

It should also be clarified that these categories of pain are not mutually exclusive and that a patient may experience various different types of pain simultaneously. In fact, in the event of a major injury, cancer or an infectious disease, the lesion may extend to both the tissues and the nerves present in the organ.

Nociceptive pain generally responds easily to conventional analgesic treatments. The World Health Organization classifies analgesics into three categories on the basis of the potency thereof. A substance that serves to reduce pain is classified as an analgesic.

Level I analgesic substances are intended for treating mild to moderate pain and include aspirin, paracetamol, and non-steroidal anti-inflammatory drugs (NSAIDs) such as ibuprofen, ketoprofen, naproxen, alminoprofen, aceclofenac, mefenamic acid, niflumic acid, tiaprofenic acid, celecoxib, dexketoprofen, diclofenac, etodolac, etoricoxib, fenoprofen, flurbiprofen, indomethacin, meloxicam, nabumetone, piroxicam, sulindac and tenoxicam. NSAIDs are referred to as such in order to distinguish them from steroidal anti-inflammatory drugs or corticosteroids, derived from cortisol, one of the hormones released during the stress response. By way of example of a corticosteroid, mention may be made of betamethasone, ciprofloxacin, cortivazol, dexamethasone, fludrocortisone, methylprednisolone, prednisolone and triamcinolone.

Level II analgesic substances are intended for treating moderate or severe pain or pain for which level I analgesics provide insufficient relief, they include codeine, dihydrocodeine, and tramadol, either alone or combined with aspirin or paracetamol.

Level III analgesic substances are intended for treating severe pain that is resistant to other analgesics and include morphine and other opium derivatives such as buprenorphine, fentanyl, hydromorphone, nalbuphine, oxycodone, and pethidine.

However, none of these treatments are free from adverse side effects. For example the use of NSAIDs can cause various adverse effects such as haemorrhages, asthma, kidney problems and more frequently gastric disorders and ulcers. Paracetamol may lead to liver toxicity. Aspirin thins the blood and attacks the stomach. Corticosteroids lead to weight gain, the weakening of immune defences, weakening of bones, and a corticosteroid dependence that results in reduced effectiveness thereof. Codeine, dihydrocodeine, and tramadol induce various adverse effects, the most common among these being nausea, vomiting, constipation, drowsiness, and drug dependence. As for morphine and opium derivatives, they induce significant adverse effects and in particular a high risk of physical and psychological dependence. Furthermore, an overdose of morphine blocks the respiratory muscles and could prove to be fatal.

Finally, certain patients may develop allergies to conventional analgesics.

There therefore exists a need to develop new compositions for the treatment and/or prevention of pain which serve to reduce the drawbacks of the analgesics of the prior art.

### SUMMARY

These objectives are achieved thanks to nicotinamide mononucleotide (NMN) and compositions that comprise the same, for use thereof in the prevention and/or treatment of pain.

The present invention relates to nicotinamide mononucleotide (NMN), a pharmaceutically acceptable precursor thereof, a pharmaceutically acceptable derivative thereof, or a pharmaceutically acceptable salt thereof, for the use thereof in the prevention and/or treatment of pain.

Advantageously, the NMN is used in an amount comprised between 0.01 mg/kg/day and 1000 mg/kg/day, preferably between 1 mg/kg/day and 100 mg/kg/day, in a more preferred manner between 5 mg/kg/day and 50 mg/kg/day, in an even more preferred manner between 10 mg/kg/day and 20 mg/kg/day.

In one embodiment, the NMN derivative may be selected from among alpha nicotinamide mononucleotide (α-NMN), dihydronicotinamide mononucleotide (denoted as NMN-H), the compound having the formula (I):

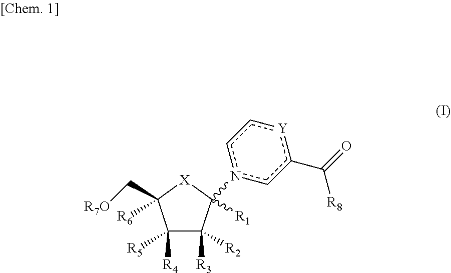

or a stereoisomer thereof, a salt thereof, a hydrate thereof, a solvate thereof, or a pharmaceutically acceptable crystal thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄, and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R₉R₁₀, and P(S)R₉R₁₀; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO₂; wherein each of R₁₁ₐ is independently selected from H and a (C₁-C₆) alkyl, and R_{11b} is independently selected from OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl,
(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₆-C₁₀) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are selected independently of one another, from among H, (C₁-C₈) alkyl, (C₁-C₈) alkyl aryl, and —CR_{B}R_{C}—C(O) —OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃;
[symbol] represents a single or a double bond along Y; and
[symbol] represents the alpha or beta anomer depending on the position of R₁;
or a stereoisomer thereof, a salt thereof, a hydrate thereof, a solvate thereof, or a crystal thereof;
or
   the compound having the formula (II):

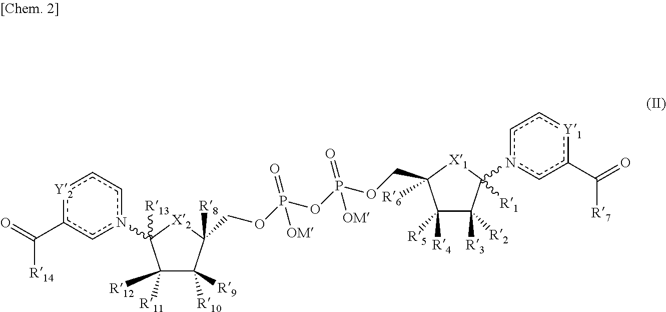

or a stereoisomer thereof, a salt thereof, a hydrate thereof, a solvate thereof, or a crystal thereof; in which
X′₁ and X′₂ are independently selected from among O, CH₂, S, Se, CHF, CF₂, and C═CH₂;
R′₁ and R′13 are independently selected from among H, azido, cyano, a C1-C8 alkyl, a C1-C8 thio-alkyl, a C1-C8 heteroalkyl, and OR, wherein R is selected from H and a C1-C8 alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and halogen; wherein R and R′ are independently selected from H and un (C₁-C₈) alkyl aryl;
Y′₁ and Y′₂ are independently selected from among CH, CH₂, C(CH₃)₂, or CCH₃;
M′ is selected from H or a suitable counter ion;
[symbol] represents a single or double bond depending on Y′₁ and Y′₂; and
[symbol] represents an alpha or beta anomer depending on the position of R′₁ and R′₁₃;
and combinations thereof.

In a preferred first embodiment, the pharmaceutically acceptable derivative is the compound having the formula (I).

In one variant of the first embodiment, X represents an oxygen.

In one variant of the first embodiment, R₁ and R₆ each independently of one another represent a hydrogen.

In one variant of the first embodiment, R₂, R₃, R₄ and R₅ each independently of one another represent a hydrogen or an OH.

In one variant of the first embodiment, Y represents a CH.

In one variant of the first embodiment, Y represents a CH₂.

In one variant of the first embodiment, R₇ represents a hydrogen.

In one variant of the first embodiment, R₇ represents P(O)(OH)₂.

In one variant of the first embodiment,
X represents an oxygen; and/or
R₁ and R₆ each independently represent a hydrogen; and/or
R₂, R₃, R₄ and R₅ each independently represent a hydrogen, or R₂, R₃, R₄ and R₅ independently represent OH; and/or
Y represents a CH or a CH₂; and/or
R₇ represents P(O)R₉R₁₀, wherein R₉ and R₁₀ are independently selected from among OH, OR₁₁, NHR₁₃, NR₁₃R₁₄, C₁-C₈ alkyl, C₂-C₈ alkenyl, C₂-C₈ alkynyl, C₃-C₁ cycloalkyl, C₅-C₁₂ aryl, C₁-C₈ aryl alkyl, C₁-C₈ alkyl aryl, C₁-C₈ heteroalkyl, C₁-C₈ heterocycloalkyl, heteroaryl, and NHCR_{A}R_{A′}C(O)R₁₂.

In one particularly preferred variant of the first embodiment, the compound of the invention is selected from among the compounds having the formula I-A to I-H:

TABLE 1

| Compounds No | |
|---|---|
| (Anomers) | Structure |
| I-A (beta) | 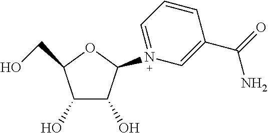 |
| I-B (alpha) | 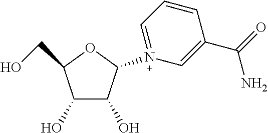 |
| I-C (beta) | 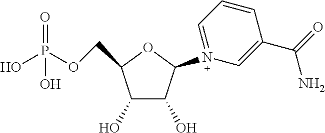 |
| I-D (alpha) | 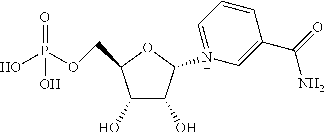 |
| I-E (beta) | 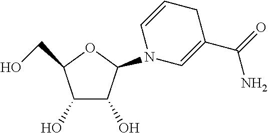 [SMILES: NC(=O)C1=CN([C@@H]2O[C@H](CO)[C@@H](O)[C@H]2O)C=CC1] |
| I-F (alpha) | 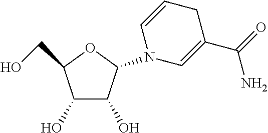 [SMILES: NC(=O)C1=CN([C@H]2O[C@H](CO)[C@@H](O)[C@H]2O)C=CC1] |
| I-G (beta) | 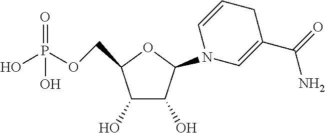 [SMILES: NC(=O)C1=CN([C@@H]2O[C@H](COP(=O)(O)O)[C@@H](O)[C@H]2O)C=CC1] |
| I-H (alpha) | 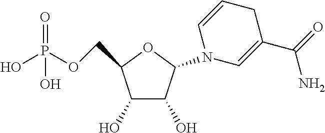 [SMILES: NC(=O)C1=CN([C@H]2O[C@H](COP(=O)(O)O)[C@@H](O)[C@H]2O)C=CC1] |

In a preferred second embodiment, the pharmaceutically acceptable derivative is the compound having the formula (II).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11, and R′12 each independently represent a hydrogen.

In one variant of the second embodiment, R′2, R′3, R′4, R′5, R′9, R′10, R′ 11, each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound according to the invention is selected from among the compounds having the formula II-A to II-F:

TABLE 2

| Compound No (Anomer) | Structure |
|---|---|
| II-A (bêta, bêta) | 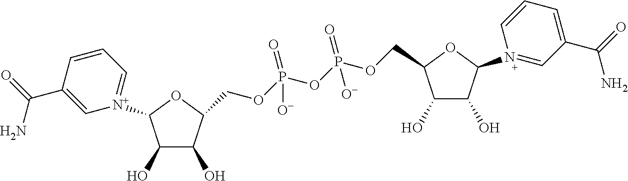 |
| II-B (bêta, alpha) | 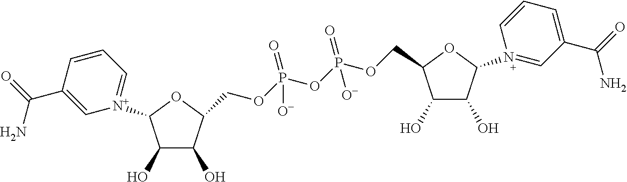 |
| II-C (alpha, alpha) | 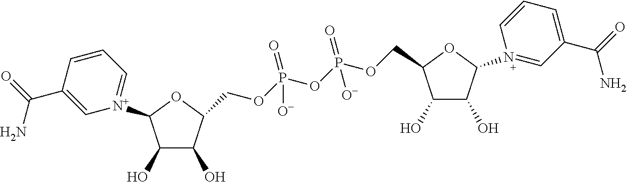 |
| II-D (bêta, bêta) | 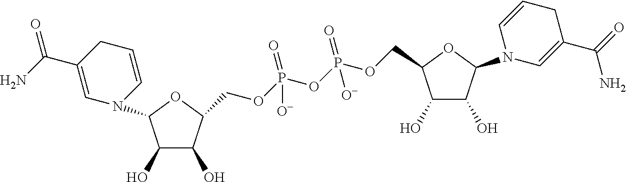 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| II-E (bêta, alpha) | 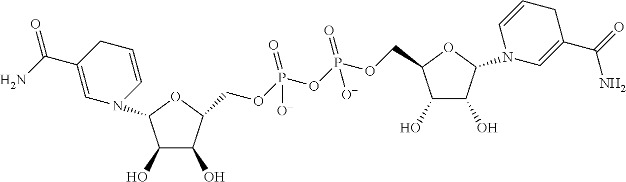 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| II-F (alpha, alpha) | 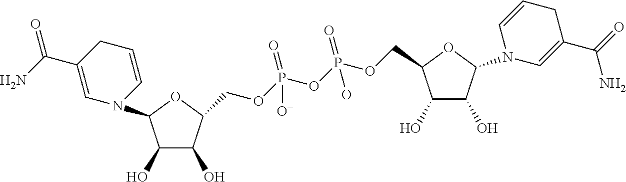 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |

In one variant of the preferred first embodiment, the pharmaceutically acceptable derivative is alpha- NMN having the formula:

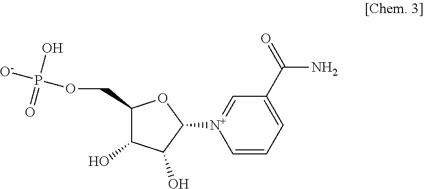 [SMILES: NC(=O)c1ccc[n+]([C@H]2O[C@H](COP(=O)([O-])O)[C@@H](O)[C@H]2O)c1]

In a preferred fourth embodiment, the pharmaceutically acceptable derivative is NMN—H:

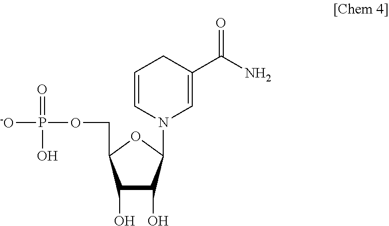

Advantageously, the pharmaceutically acceptable precursor is nicotinamide riboside (denoted NR):

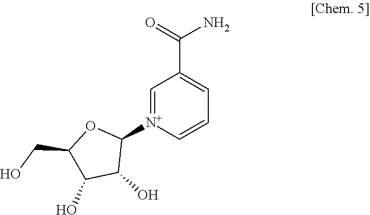 [SMILES: NC(=O)c1ccc[n+]([C@@H]2O[C@H](CO)[C@@H](O)[C@H]2O)c1]

or dihydronicotinamide riboside (denoted —NR—H) having the formula:

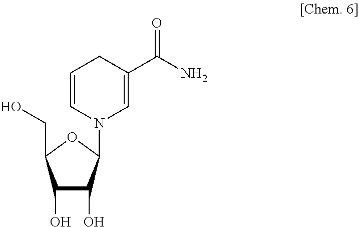

Advantageously, the nicotinamide mononucleotide (NMN), a pharmaceutically acceptable precursor thereof, a pharmaceutically acceptable derivative thereof, or a pharmaceutically acceptable salt thereof, may be administered via various routes: oral, ocular, sublingual, parenteral, transcutaneous, vaginal, epidural, intravesical, rectal, or inhalation.

Preferably, the NMN, a pharmaceutically acceptable precursor thereof, a pharmaceutically acceptable derivative thereof, or a pharmaceutically acceptable salt thereof, is administered via the oral or parenteral routes.

Advantageously, the NMN, a pharmaceutically acceptable precursor thereof, a pharmaceutically acceptable derivative thereof, or a pharmaceutically acceptable salt thereof, is used in the treatment and/or prevention of pain in mammals, preferably humans.

In one preferred embodiment, the pain is a nociceptive pain.

In one preferred embodiment, the pain is not a neuropathic pain.

Advantageously, the NMN, a pharmaceutically acceptable precursor thereof, a pharmaceutically acceptable derivative thereof, or a pharmaceutically acceptable salt thereof, is used in order to reduce allodynia.

Advantageously, the NMN, a pharmaceutically acceptable precursor thereof, a pharmaceutically acceptable derivative thereof, or a pharmaceutically acceptable salt thereof, is used in order to reduce hyperalgesia.

Advantageously, the pain is a visceral pain.

Advantageously, the pain is a pain in the urogenital (or genitourinary) system.

Advantageously, the pain is a pain caused by a urinary tract infection.

Advantageously, the NMN, a pharmaceutically acceptable precursor thereof, a pharmaceutically acceptable derivative thereof, or a pharmaceutically acceptable salt thereof, is used in combination with at least one other therapeutic agent.

Advantageously, the at least one additional therapeutic agent is selected from among antibiotics, antifungals, antivirals, and combinations thereof.

Advantageously, the at least one therapeutic agent is an analgesic.

Advantageously, the analgesic is selected from among paracetamol, aspirin, non-steroidal anti-inflammatories, cortisone derivatives, and combinations thereof.

Advantageously, the non-steroidal anti-inflammatory is selected from among ibuprofen, ketoprofen, naproxen, alminoprofen, aceclofenac, mefenamic acid, niflumic acid, tiaprofenic acid, celecoxib, dexketoprofen, diclofenac, etodolac, etoricoxib, fenoprofen, flurbiprofen, indomethacin, meloxicam, nabumetone, piroxicam, sulindac, tenoxicam, and combinations thereof.

Advantageously, the cortisone derivative is selected from among betamethasone, ciprofloxacin, cortivazol, dexamethasone, fludrocortisone, methylprednisolone, prednisolone, and triamcinolone, and combinations thereof.

Advantageously, the analgesic is selected from among codeine, dihydrocodeine, tramadol, and combinations thereof.

Advantageously, the analgesic is selected from among morphine, buprenorphine, fentanyl, hydromorphone, nalbuphine, oxycodone, pethidine and combinations thereof.

The present invention also relates to a composition comprising nicotinamide mononucleotide, a pharmaceutically acceptable precursor thereof, a pharmaceutically acceptable derivative thereof, or a pharmaceutically acceptable salt thereof, and at least one pharmaceutically acceptable excipient, for the use thereof in the prevention and/or treatment of pain as described above.

Advantageously, the composition according to the invention is in the form of a tablet, a capsule, a sachet, a granule, a soft capsule, a lozenge, a lyophilisate, a suspension, a gel, a syrup, a solution, a water/oil emulsion, an oil/water emulsion, an oil, a cream, a milk, a spray, an ointment, an ampoule, a suppository, an eye drop, a vaginal ovule, a vaginal capsule, a liquid for inhalation, a dry powder inhaler, a pressurised metered dose inhaler.

Preferably, the composition according to the invention is in the form of a gastro-resistant capsule or a sublingual tablet.

Advantageously, the composition according to the invention is a pharmaceutical composition.

Advantageously, the composition according to the invention is a dietary supplement.

The present invention also relates to a composition comprising nicotinamide mononucleotide, a pharmaceutically acceptable derivative thereof, or a pharmaceutically acceptable salt thereof, at least one pharmaceutically acceptable excipient, and at least one additional therapeutic agent, for the use thereof in the prevention and/or the treatment of pain as described above.

### Definitions

In the present invention, the following terms have the following meaning.

Unless otherwise indicated, the nomenclature of substituents which are not explicitly defined in the present invention is obtained by naming the terminal portion of the functional group followed by the adjacent functional group towards the point of attachment.

“Alkyl” by itself or as part of another substituent refers to a hydrocarbyl radical having the formula CnH2n+1 in which n is a number greater than or equal to 1. In general, the alkyl groups of this invention include from 1 to 12 carbon atoms, preferably from 1 to 8 carbon atoms, more preferably from 1 to 6 carbon atoms, even more preferably from 1 to 2 carbon atoms. The alkyl groups may be linear or branched and may be substituted as indicated in the present invention. The alkyls that are suitable for the purposes of implementation of the invention may be selected from among methyl, ethyl, n-propyl, i-propyl, n-butyl, i-butyl, s-butyl and t-butyl; pentyl and its isomers such as n-pentyl and iso-pentyl; and hexyl and its isomers such as n-hexyl and iso-hexyl; heptyl and its isomers (for example n-heptyl, iso-heptyl); octyl and its isomers (for example n-octyl, iso-octyl); nonyl and its isomers (for example n-nonyl, iso-nonyl); decyl and its isomers (for example n-decyl, iso-decyl); undecyl and its isomers; dodecyl and its isomers. Preferably, the alkyl groups may be selected from among methyl, ethyl, n-propyl, i-propyl, n-butyl, i-butyl, s-butyl, t-butyl, n-pentyl, n-hexyl, n-heptyl, n-octyl, n-nonyl, and n-decyl. The saturated and branched alkyl groups may be selected, without limitation, from among isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, and 3,3-diethylhexyl. The preferred alkyl groups are the following: methyl, ethyl, n-propyl, i-propyl, n-butyl, i-butyl, s-butyl and t-butyl. Cx-Cy-alkyls refer to alkyl groups that contain from x to y carbon atoms.

When the suffix “ene” (“alkylene”) is used in conjunction with an alkyl group, it indicates that the alkyl group as defined herein has two single bonds as points of attachment to other groups. The term “alkylene” includes methylene, ethylene, methylmethylene, propylene, ethylethylene, and 1,2-dimethylethylene.

The term “alkenyl” as used herein refers to an unsaturated hydrocarbyl group, which may be linear or branched, that comprises one or more carbon-carbon double bonds. The alkenyl groups that are suitable comprise between 2 and 12 carbon atoms, preferably between 2 and 8 carbon atoms, and even more preferably between 2 and 6 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and other similar groups.

The term “alkynyl”, as used herein, refers to a class of monovalent unsaturated hydrocarbyl groups, in which the unsaturation results from the presence of one or more carbon-carbon triple bond(s). The alkynyl groups generally, and preferably, have the same number of carbon atoms as described here above for the alkenyl groups. Without limitation, some examples of alkynyl groups include ethynyl, 2-propynyl, 2-butynyl, 3-butynyl, 2-pentynyl and its isomers, 2-hexynyl and its isomers, etc.

“Alkoxy” refers to an alkyl group as defined here above, which is attached to another moiety by means of an oxygen atom. Examples of alkoxy groups include the groups: methoxy, isopropoxy, ethoxy, tert-butoxy, and the like. The alkoxy groups may be optionally substituted by one or more substituent(s). The alkoxy groups included in the compounds of this invention may be optionally substituted with a solubilising group.

“Aryl”, as used herein, refers to a polyunsaturated aromatic hydrocarbyl group having a single ring (for example phenyl) or multiple aromatic rings that are fused together (for example naphthyl) or covalently bonded, which generally contains 5 to 18 atoms, preferably 5 to 12, in a more preferred manner 6 to 10, with at least one of the said rings being aromatic. The aromatic ring may optionally include one or two additional rings (cycloalkyl, heterocyclyl, or heteroaryl) fused thereto. The aryl is also intended to include partially hydrogenated derivatives of the carbocyclic systems listed herein. Examples of aryl include phenyl, biphenylyl, biphenylenyl, 5- or 6-tetralinyl; naphthalene-1- or -2-yl; 4-, 5-, 6 or 7-indenyl; 1-, 2-, 3-, 4-, or 5-acenaphthylenyl; 3-, 4-, or 5-acenaphthenyl; 1-, or 2-pentalenyl; 4-, or 5-indanyl; 5-, 6-, 7-, or 8-tetrahydronaphthyl; 1,2,3,4-tetrahydronaphthyl; 1,4-dihydronaphthyl; 1-, 2-, 3-, 4-, or 5-pyrenyl.

When at least one carbon atom in an aryl group is replaced by a heteroatom, the resulting ring is referred to herein as a “heteroaryl” ring.

“Alkylaryl” refers to an aryl group substituted by an alkyl group.

“Amino acid” refers to an alpha-amino carboxylic acid, that is to say, a molecule comprising a carboxylic acid functional group and an amino functional group in the alpha position of the carboxylic acid group, for example a proteinogenic amino acid or a non-proteinogenic amino acid.

“Proteinogenic amino acid” refers to an amino acid that is incorporated into the proteins during the translation of the messenger RNA by the ribosomes in living organisms, that is to say, Alanine (ALA), Arginine (ARG), Asparagine (ASN), Aspartate (ASP), Cysteine (CYS), Glutamate (glutamic acid) (GLU), Glutamine (GLN), Glycine (GLY), Histidine (HIS), Isoleucine (ILE), Leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophan (TRP), Tyrosine (TYR), or Valine (VAL).

“Non-proteinogenic amino acid” as used herein refers to an amino acid that is not naturally encoded or found in the genetic code of a living organism. Without limitation, some examples of non-proteinogenic amino acid are: ornithine, citrulline, argininosuccinate, homoserine, homocysteine, cysteine-sulfinic acid, 2-aminomuconic acid, δ-aminolevulinic acid, β-alanine, cystathionine, γ-aminobutyrate, dihydroxyphenylalanine (DOPA), 5-hydroxytryptophan, D-serine, ibotenic acid, α-aminobutyrate, 2-aminoisobutyrate, D-leucine, D-valine, D-alanine, and D-glutamate.

The term “cycloalkyl” as used herein refers to a cyclic alkyl group, that is to say, a monovalent, saturated or unsaturated hydrocarbyl group, having 1 or 2 ring structures. The term “cycloalkyl” includes monocyclic or bicyclic hydrocarbyl groups. The cycloalkyl groups may comprise 3 or more carbon atom(s) in the ring and generally, according to the present invention, comprise from 3 to 10, more preferably from 3 to 8 carbon atoms, and even more preferably from 3 to 6 carbon atoms. Examples of cycloalkyl groups include, but are not limited to, cyclopropyl, cyclobutyl, cyclopentyl, cyclohexyl, with cyclopropyl being particularly preferred.

The term “pharmaceutically acceptable excipient” refers to an inert carrier or support substance used as a solvent or diluent within which the active ingredient is formulated and/or administered, and which does not produce an adverse, allergic or other reaction when it is administered to an animal, preferably to a human. This includes all solvents, dispersing media, coatings, antibacterial and antifungal agents, isotonic agents, absorption retardants, and other similar ingredients. For human administration, the preparations must meet specific standards of sterility, general safety and purity, as required by the regulatory authorities, such as for example the Food and Drug Administration (FDA) in the United States of America, or the European Medicines Agency (EMA). Within the meaning of the invention, “pharmaceutically acceptable excipient” includes all pharmaceutically acceptable excipients as well as all pharmaceutically acceptable carriers, diluents and/or adjuvants.

“Halogen” or “halo” refers to fluoro, chloro, bromo or iodo. The preferred halo groups are fluoro and chloro.

“Haloalkyl” alone or in combination, refers to an alkyl radical having the meaning as defined here above, in which one or more hydrogen atom(s) are replaced by a halogen as defined here above. By way of examples of such haloalkyl radicals, the following may be cited: chloromethyl, 1-bromoethyl, fluoromethyl, difluoromethyl, trifluoromethyl, 1,1,1-trifluoroethyl, and similar radicals. ‘Cx-Cy-haloalkyl’ and ‘Cx-Cy-alkyl’refer to alkyl groups that contain from x to y carbon atoms. The preferred haloalkyl groups are difluoromethyl and trifluoromethyl.

“Heteroalkyl” refers to an alkyl group as defined here above, in which one or more carbon atom(s) are replaced by a heteroatom selected from among oxygen, nitrogen and sulfur atoms. In the heteroalkyl groups, the heteroatoms are bonded along the alkyl chain only to carbon atoms, that is to say, each heteroatom is separated from every other heteroatom by at least one carbon atom. However, the nitrogen and sulfur heteroatoms may optionally be oxidised and the nitrogen heteroatoms may optionally be quaternised. A heteroalkyl is bonded to another group or molecule only by means of a carbon atom, that is to say, the bonding atom is not selected from the heteroatoms included in the heteroalkyl group.

The term “heteroaryl” as used herein, alone or as part of another group, refers to, but is not limited to, aromatic rings of 5 to 12 carbon atoms or ring systems containing 1 or 2 rings that are fused or covalently bonded, and generally containing 5 or 6 atoms, with at least one of the said rings being aromatic; in which one or more carbon atom(s) in one or more of these rings are replaced by oxygen, nitrogen and/or sulfur atoms, it being possible for the nitrogen and sulfur heteroatoms to optionally be oxidised and for the nitrogen heteroatoms to optionally be quaternised. These rings may be fused to an aryl, cycloalkyl, heteroaryl or heterocyclyl ring. Without limitation, some examples of such heteroaryls include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, dioxinyl, thiazinyl, triazinyl, imidazo [2,1-b][1,3]thiazolyl, thieno [3,2-b]furanyl, thieno [3,2-b]thiophenyl, thieno [2,3-d][1,3]thiazolyl, thieno [2,3-d]imidazolyl, tetrazolo [1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1, 2,3-benzothiadiazolyl, 2,1,3-benzothiadiazolyl, thienopyridinyl, purinyl, imidazo [1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

When at least one carbon atom in a cycloalkyl group is replaced by a heteroatom, the resulting ring is referred to herein as “heterocycloalkyl” or “heterocyclyl”.

The terms “heterocyclyl”, “heterocycloalkyl”, or “heterocyclo”, as used herein by themselves or as part of another group, refer to non-aromatic cyclic groups, either fully saturated or partially unsaturated (for example, 3 to 7 membered monocyclic, 7 to 11 membered bicyclic groups or containing a total of 3 to 10 ring atoms), which have at least one heteroatom in at least one ring containing a carbon atom. Each ring of the heterocyclic group containing a heteroatom may have 1, 2, 3, or 4 heteroatoms selected from among nitrogen, oxygen and/or sulfur atoms, where the nitrogen and sulfur heteroatoms may optionally be oxidised, and the nitrogen heteroatoms may optionally be quaternised. Any whichever of the carbon atoms of the heterocyclic group may be substituted by an oxo (for example piperidone, pyrrolidinone). The heterocyclic group may be attached to any heteroatom or carbon atom in the ring or ring system, where the valence so permits. The rings of multi-ring heterocycles may be fused, bridged and/or joined/connected by one or more spiro atoms. Exemplary heterocyclic groups include, but are not limited to, the following groups: oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholine-4-yl, thiomorpholine-4-ylsulf oxide, thiomorpholine-4-ylsulfone, 1,3-dioxolanyl, 1,4-oxathianyl, 1H-pyrrolizinyl, tetrahydro-1,1-dioxothiophenyl, N-formylpiperazinyl, and morpholine-4-yl.

The term “precursor” as used herein also refers to pharmacologically acceptable derivatives of compounds having the formula (I) or (II) such as esters, of which the in vivo biotransformation product is the active drug. Precursors are characterised by increased bioavailability and are readily metabolised into active compounds in vivo. The precursors that are appropriate for the purposes of the invention include in particular carboxylic esters, in particular alkyl esters, aryl esters, acyloxyalkyl esters, and the carboxylic esters of dioxolene; ascorbic acid esters.

“Pharmaceutically acceptable” refers to the state of being approved, or with the likelihood of being potentially approved by a regulatory body or listed in a recognised pharmacopoeia for use in animals, and more preferably in humans. It may pertain to a substance that is not biologically or otherwise undesirable; that is to say, the substance may be administered to an individual without causing adverse biological effects or deleterious interactions with one of the components of the composition within which it is contained. Preferably, a “pharmaceutically acceptable” salt or excipient refers to any salt or any excipient that is authorised by the European Pharmacopoeia (denoted as “Ph. Eur.”) and the American Pharmacopoeia (referred to as “United States Pharmacopeia (USP)” in English).

The term “active ingredient” refers to a molecule or a substance which when administered to a subject slows down or stops the progression, aggravation or deterioration of one or more symptom(s) of a disease or a condition; relieves the symptoms of a disease or a condition; cures a disease or a condition. According to one of these embodiments, the therapeutic ingredient is a small molecule, which is natural or synthetic. According to another embodiment, the therapeutic ingredient is a biological molecule such as, for example, an oligonucleotide, a small interfering RNA (siRNA), a microRNA (miRNA), a DNA fragment, an aptamer, an antibody and the like. “Pharmaceutically acceptable salts” include the acid addition salts and base addition salts of these said salts. Suitable acid addition salts are formed from acids that form non-toxic salts. Examples that may be cited include: acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, and salts of xinofoate. Suitable basic salts are formed from bases which form non-toxic salts. By way of examples, mention may be made of the salts of: aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine, and zinc. Hemisalts of acids and bases may also be formed, for example, hemisulfates and salts of chemical calcium. The preferred pharmaceutically acceptable salts are hydrochloride/chloride, bromide/hydrobromide, bisulfate/sulfate, nitrate, citrate and acetate.

Pharmaceutically acceptable salts may be prepared by one or more of the following methods:
i. by reacting the compound with the desired acid;
ii. by reacting the compound with the desired base;
iii. by removing an acid or base labile protecting group under basic or acidic conditions from a suitable precursor of the compound, or by ring opening of a suitable cyclic precursor, for example a lactone or a lactam, using the desired acid; or
iv. by converting one salt of the compound into another by reacting the initial salt with an appropriate acid or by means of an appropriate ion exchange column.

All of these reactions are generally carried out in solution. The salt can precipitate out of the solution and may be collected by filtration or may be recovered by evaporation of the solvent. The degree of ionisation of the salt may vary from completely ionised to almost non-ionised.

“Solvate” is used herein to describe a molecular complex that comprises the compound of the invention and one or more pharmaceutically acceptable solvent molecules, for example, ethanol.

The term “substituent” or “substituted” indicates that a hydrogen radical on a compound or a group is replaced by any desired group which is substantially stable under the reaction conditions in an unprotected form or when it is protected by a protecting group. Examples of preferred substituents include, but are not limited to: a halogen (chloro, iodo, bromo, or fluoro); an alkyl; an alkenyl; an alkynyl, as described here above; a hydroxy; an alkoxy; a nitro; a thiol; a thioether; an imine; a cyano; an amido; a phosphonato; a phosphine; a carboxyl; a thiocarbonyl; a sulfonyl; a sulfonamide; a ketone; an aldehyde; an ester; an oxygen (—O); a haloalkyl (for example, trifluoromethyl); a cycloalkyl, which may be condensed-ring or non-condensed-ring monocyclic or polycyclic (for example, cyclopropyl, cyclobutyl, cyclopentyl or cyclohexyl); or a heterocycloalkyl, which may be condensed-ring or non-condensed-ring monocyclic or polycyclic (for example, pyrrolidinyl, piperidinyl, piperazinyl, morpholinyl, or thiazinyl); fused or unfused monocyclic or polycyclic, aryl or heteroaryl (for example, aryl, heteroaryl, pyrrolidinyl, piperidinyl, piperazinyl, morpholinyl, or thiazinyl); fused or unfused monocyclic or polycyclic (for example, aryl, heteroaryl, pyrrolidinyl, piperidinyl, piperazinyl, morpholinyl, or thiazinyl), phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazidinyl, pyridaziminyl, pyridaziminyl, benzimidazolyl, benzothiophenyl, or benzofuranyl); amino (primary, secondary or tertiary); CO₂CH₃; CONH₂; OCH₂CONH₂; NH₂; SO₂NH₂ OCHF₂; FC₃; OCF₃; moreover these groups may also be optionally substituted by a fused ring bridge or structure, for example —OCH₂O—. These substituents may optionally be further substituted by a substituent selected from among these groups. In certain representations, the term “substituent” or the adjective “substituted” refers to a substituent selected from the group constituted of: an alkyl, an alkenyl, an alkynyl, a cycloalkyl, a cycloalkenyl, a heterocycloalkyl, an aryl, a heteroaryl, an arylalkyl, a heteroarylalkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR′₁₃, —OC(O)R₁₃, —NR₁₃C(O)NR₁₁R₁₂, —OC(O)NR₁₁R₁₂, —NR₁₃C(O)OR₁₄, —S(O)rR13, —NR₁₃S(O)rR₁₄, —OS(O)rR₁₄, S(O)rNR₁₁R₁₂, —O, —S, and —NR₁₃, where r is 1 or 2; R₁₁ and R₁₂, for each occurrence, are independently H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₁ and R₁₂ taken together with the nitrogen to which they are attached are an optionally substituted heterocycloalkyl, or an optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In certain variats, the term “substituent” or the adjective “substituted” refers to a solubilising group.

The term “administration”, or a variant of this term (for example, “administer”), refers to the providing of the active ingredient, whether alone or as part of a pharmaceutically acceptable composition, to the patient who is to receive the same in the context of treatment or prevention of a condition, a symptom, or a disease.

The terms “treating”, “curing”, and “treatment”, as used herein, are meant to include the relieving, alleviation, or ablation of a condition, or a disease and/or the symptoms associated therewith.

The terms “prevent”, “impede” and “prevention”, as used in the present invention, refer to a method that serves the purpose of: delaying, or impeding or preventing the onset of a condition, or a disease and/or the symptoms associated therewith; preventing a patient from contracting a condition or a disease; or reducing the risk of a patient's contracting a given disease or a condition.

The bonds of an asymmetric carbon may be represented herein using a solid triangle ( [symbol] ), a dotted triangle ( [symbol] ), or a zigzag line ( [symbol] ).

### FIGURES

FIG. 1A is a graph showing the nociceptive score induced by the administration of cyclophosphamide in rats versus animals treated with the carrier.

FIG. 1B is a graph showing the nociceptive threshold induced by the administration of cyclophosphamide in rats versus animals treated with the carrier.

FIG. 2A is a graph showing the evolution of the nociceptive threshold in the rats, 2 hours and 4 hours after the administration of cyclophosphamide as compared with the carrier.

FIG. 2B is a graph showing the evolution of the nociceptive score in the rats, 2 hours and 4 hours after the administration of cyclophosphamide as compared with the carrier.

FIG. 3A shows the nociceptive threshold in animals treated with cyclophosphamide to whom the carrier, the NMN, or the ibuprofen was administered at TO after administration of cyclophosphamide.

FIG. 3B shows the nociceptive threshold in animals treated with cyclophosphamide to whom the carrier, the NMN, or the ibuprofen was administered at T=2 h after administration of cyclophosphamide.

FIG. 3C shows the nociceptive threshold in animals treated with cyclophosphamide to whom the carrier, the NMN, or the ibuprofen was administered at T=4 h after administration of cyclophosphamide.

FIG. 4A shows the nociceptive score in the animals treated with cyclophosphamide to whom the carrier, the NMN, or the ibuprofen was administered at TO after the administration of cyclophosphamide.

FIG. 4B shows the nociceptive score in the animals treated with cyclophosphamide to whom the carrier, the NMN, or the ibuprofen was administered at T0, T=2 h, or T=4 h after the administration of cyclophosphamide.

FIG. 4C shows the nociceptive score in the animals treated with cyclophosphamide to whom the carrier, the NMN, or the ibuprofen was administered at T=4 h after the administration of cyclophosphamide.

FIG. 5A is a graph showing the baseline nociceptive threshold for each experimental group.

FIG. 5B is a graph showing baseline nociceptive scores for all of the experimental groups.

FIG. 6A is a graph showing the nociception threshold for the effects of NMN, Compound A, and Compound B on allodynia induced by CYP at 2 hours.

FIG. 6B is a graph showing the nociceptive threshold for the effects of NMN, Compound A, and Compound B on allodynia induced by CYP at 4 hours.

FIG. 7A is a graph showing the effects of NMN on visceral pain induced by CYP at 2 hours (nociceptive scores).

FIG. 7B is a graph showing the effects of NMN on Visceral pain induced by CYP at 4 hours (nociceptive scores).

FIG. 8A is a graph showing the effects of Compound A on visceral pain induced by CYP at 2 h (nociceptive scores).

FIG. 8B is a graph showing the effects of Compound A on Visceral pain induced by CYP at 4 h (nociceptive scores).

FIG. 9A is a graph showing the effects of Compound B on Visceral pain induced by CYP at 2 h (nociceptive scores).

FIG. 9B is a graph showing the effects of Compound B on visceral pain induced by CYP at 2 h (nociceptive scores).

### DETAILED DESCRIPTION

The subject matter of the present invention relates to nicotinamide mononucleotide (NMN), a pharmaceutically acceptable precursor thereof, a pharmaceutically acceptable derivative thereof, or a pharmaceutically acceptable salt thereof, for the use thereof in the prevention and/or treatment of pain.

The subject matter of the present invention also relates to a composition comprising nicotinamide mononucleotide (NMN), a pharmaceutically acceptable precursor thereof, a pharmaceutically acceptable derivative thereof, or a pharmaceutically acceptable salt thereof, and at least one pharmaceutically acceptable excipient, for the use thereof in the prevention and/or treatment of pain.

Nicotinamide adenine dinucleotide (NAD) is a coenzyme present in all living cells. NAD exists in the cell either in its oxidised form NAD+, or in its reduced form NADH. The role of NAD is that of an electron carrier that is involved in the oxidation-reduction reactions of metabolism. NAD is moreover also involved in a number of cellular processes such as adenosine diphosphate (ADP) ribosylation in the context of post-translational modifications of proteins.

NAD may be synthesised de novo by the cell from amino acids such as tryptophan or aspartate. However, such synthesis is marginal because the main pathway for NAD synthesis is the salvage pathway, by means of which the cell, and primarily the cell nucleus, recycles compounds in order to reform NAD from precursors. The precursors of NAD include niacin, nicotinamide riboside, nicotinamide mononucleotide, and nicotinamide.

NMN is one of the compounds that enable the synthesis of NAD by the salvage pathway and has the formula:

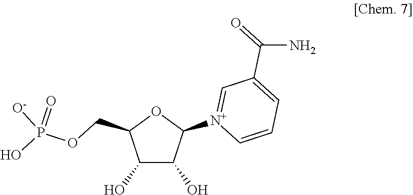 [SMILES: NC(=O)c1ccc[n+]([C@@H]2O[C@H](COP(=O)([O-])O)[C@@H](O)[C@H]2O)c1]

The inventors have indeed demonstrated that the use of NMN, a pharmaceutically acceptable precursor thereof, a pharmaceutically acceptable derivative thereof, or a pharmaceutically acceptable salt thereof, and of the composition according to the invention makes it possible to obtain an effect on pain that is comparable to the medicinal products commonly used for treating pain. More precisely, the use of NMN and of the composition according to the invention, in accordance with the invention, provides the ability to prevent pain because it provides the means to reduce the intensity of the pain developed in response to a painful stimulus. The use of NMN, a pharmaceutically acceptable precursor thereof, a pharmaceutically acceptable derivative thereof, or a pharmaceutically acceptable salt thereof, and of the composition according to the invention is also effective for treating pain that is already present.

In addition, the use of NMN, which is a molecule naturally present in the body, has many advantages. In particular, NMN and the composition according to the invention are well tolerated by patients. The use of NMN and of the composition according to the invention in fact does not induce any allergy in patients. In addition, the use of NMN and of the composition according to the invention does not induce the adverse side effects frequently encountered with conventional analgesics such as, for example, ulcers, hepatic toxicity, blood thinning, drowsiness, nausea, and vomiting.

Furthermore, NMN also does not induce any phenomenon of physical or psychological dependence, unlike analgesics that comprise morphine or opium derivatives. The use of NMN and of the composition according to the invention for preventing and/or treating pain is therefore safe for patients.

The NMN and the composition according to the invention may therefore be used in children and adults. NMN is indeed well tolerated by children. In the context of the invention, a patient is considered to be a child when they are less than 16 years of age, and an adult from 16 years of age onwards.

In one preferred embodiment, the NMN is in the form of a zwitterion. The term “zwitterion” is understood to refer to a molecular chemical species that possesses electrical charges of opposite signs and situated, in general, on non-adjacent atoms of the molecule.

The pharmaceutically acceptable excipient may be selected from among a bulking agent, a lubricant, a flavouring agent, a colouring agent, an emulsifier, a compression agent, a diluent, a preservative, a gelling agent, a plasticiser, a surfactant, or combinations thereof. A person skilled in the art would know to determine the excipient to be selected based on the galenic form that they would have selected.

In the context of the present invention, an “excipient” refers to any substance other than the NMN that is in the composition and has no therapeutic effect. The excipient does not interact chemically with the NMN or any other additional therapeutic agent.

The NMN, a pharmaceutically acceptable precursor thereof, a pharmaceutically acceptable derivative thereof, or a pharmaceutically acceptable salt thereof, and the composition according to the invention may be administered in a therapeutically effective amount. In the context of the invention, a therapeutically effective amount indicates that the composition is administered to a patient in an appropriate amount that is sufficient to obtain the desired therapeutic effect, in this case the reducing of the sensation of pain.

In one embodiment, the NMN, a pharmaceutically acceptable precursor thereof, a pharmaceutically acceptable derivative thereof, or a pharmaceutically acceptable salt thereof, is used in an amount comprised between 0.01 mg/kg/day and 1000 mg/kg/day, preferably between 1 mg/kg/day and 100 mg/kg/day, in a more preferred manner between 5 mg/kg/day and 50 mg/kg/day, in an even more preferred manner between 10 mg/kg/day and 20 mg/kg/day. A person skilled in the art is able to adapt the dose of NMN to be administered according to the age and weight of the patient, and the intensity of the pain to be treated.

An appropriate dosage level may be about 0.01 to 250 mg/kg per day, about 0.05 to 100 mg/kg per day, or about 0.1 to 50 mg/kg per day. Within this range, the dose may be from 0.05 to 0.5, from 0.5 to 5, or from 5 to 50 mg/kg per day. For oral administration, the compositions are preferably provided in the form of tablets containing from 1.0 to 1000 milligrammes of the active ingredient, in particular 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrammes of the active ingredient for symptom based dose adjustment for the patient to be treated. For example, the dosage may be comprised between 100 mg/day and 5000 mg/day, preferably between 500 mg/day and 1000 mg/day. The compounds may be administered based on a schedule of 1 to 4 times per day, preferably one, two, or three times per day, preferably three times per day. The duration of treatment depends on and is determined by the physician. It may range from one day to one year or even longer, preferably from one week to three months, more preferably from two weeks to six weeks. It is to be understood, however, that the specific dose level and frequency of dosing, as well as the duration for a given patient may vary and will depend on a variety of factors, in particular the potency of action of the specific compound used, the metabolic stability and the duration of action of this compound, the subject's age, body weight, general health condition, gender, diet, the mode and time of administration, rate of excretion, combination of medications, and the host being subjected to treatment.

The NMN, a pharmaceutically acceptable precursor thereof, a pharmaceutically acceptable derivative thereof, or a pharmaceutically acceptable salt thereof, may be administered at a daily dose of 10 mg/kg, with a minimum of 50 mg/day and a maximum of 1000 mg/day.

Different pain scales exist for evaluating pain, on the basis of the age of the patient and their cognitive state, and they are well known to the person skilled in the art. There are scales for:
self-assessment (self-reporting) by the patient, either adult or child from 4-6 years of age (school age), capable of communicating the intensity or characteristics of the pain,
hetero-assessment, that is to say the assessment by caregivers, of the pain of non-communicating adults (elderly persons, patients in intensive care, persons with multiple disabilities, etc), or of a child under 4 years of age.

The scales for self-reporting pain in children include in particular the vertical visual analogue (VAS) scale, the numerical (NS) scale, the face pain scale, the poker chips scale, and the body diagram on which the child situates their pain.

Hetero-assessment scales for acute pain in children include the Neonatal Facial Coding System (NFCS), the Neonatal Infant Pain Scale (NIPS) for newborns, the scale based on observation of facial expression, leg and arm movements, crying, and consolability (ie “Face, Legs, Activity, Cry, Consolability” or FLACC), the comfort scale (or “Comfort Behaviour” scale), the Premature Infant Pain Profile (or PIPP), the paediatric outpatient acute pain scale (or CHEOPS for Children's Hospital of Eastern Ontario Pain Scale”), the French paediatric pain scale ENVENDOL (Evaluation Enfant Douleur). Hetero-assessment scales for chronic pain in children include the French neonatal infant pain and discomfort scale EDIN (Evaluation de Douleur et d′Inconfort du Nouveau-né), the Objective Pain Scale, the Amiel-Tison system with inverse scoring, the Gustave-Roussy Child Pain Scale (Douleur Enfant Gustave-Roussy—DEGR), the French pediatric pain hetero-assessment scale HEDEN (Hetero Evaluation de la Douleur de l′Enfant), and the Saint-Antoine Pain Questionnaire (Questionnaire de la Douleur de Saint-Antoine—QDSA).

Adult self-assessment (self-reporting) scales include the vertical visual analogue scale, the numerical rating scale (NRS), and the simple verbal rating scale (VRS). Hetero-assessment scales in adults include the Algoplus test, the Doloplus test, and the behavioural pain assessment scale for older adults (Évaluation Comportementale Chez la Personne Âgée—ECPA). Finally, there is a specific test for the evaluation of neuropathic pain: the DN4 test (Douleur Neuropathique 4 Questions).

The NMN, a pharmaceutically acceptable precursor thereof, a pharmaceutically acceptable derivative thereof, or a pharmaceutically acceptable salt thereof, and the composition according to the invention may be administered once per day or multiple times per day. In particular, the NMN and the composition according to the invention may be administered between 1 and 12 times per day, preferably between 3 and 10 times per day, more preferably between 5 and 8 times per day.

The dose administered and the frequency of administration depend in particular on the intensity of the pain felt by the patient.

### Use

According to the present invention, the NMN, a pharmaceutically acceptable precursor thereof, a pharmaceutically acceptable derivative thereof, or a pharmaceutically acceptable salt thereof, and the composition according to the invention are used for preventing and/or treating pain. In one embodiment, the pain is not a neuropathic pain. Preferably, the pain is a nociceptive pain.

The NMN, a pharmaceutically acceptable precursor thereof, a pharmaceutically acceptable derivative thereof, or a pharmaceutically acceptable salt thereof, and the composition according to the invention are used in particular for reducing the sensitivity to pain. In one embodiment, the NMN, a pharmaceutically acceptable precursor thereof, a pharmaceutically acceptable derivative thereof, or a pharmaceutically acceptable salt thereof, and the compositions that comprise the same in accordance with the invention, are used in order to reduce allodynia. In the context of the invention, allodynia is a pain felt in response to a stimulus that normally does not cause pain. In other words, in a situation of allodynia, the pain tolerance threshold of the patient is reduced. The use of NMN, a pharmaceutically acceptable precursor thereof, a pharmaceutically acceptable derivative thereof, or a pharmaceutically acceptable salt thereof, and of compositions that comprise the same in accordance with the invention therefore makes it possible to restore a normal threshold of pain tolerance in the patient and to eliminate, or at the very least to reduce, the phenomenon of allodynia. Various different techniques that are well known to the person skilled in the art are used to assess allodynia in humans. Allodynia in humans can for example be measured by means of stimulation with different filaments (principle of the von Frey test) either on a wound (post-operative surface), or after intradermal injection of capsaicein, or after application of heat to the skin. Allodynia may also be measured using questionnaires in which the patient indicates the score for their pain after, for example, immersing their hand in ice, or after application of heat for a given time period at a given temperature to an area, or again after application of a defined pressure to different sites (arm, knee, perineum, anus, etc). Another possible way of assessing is to stimulate an area with stimuli known to be non-pain inducing and to assess the pain that the patient feels by means of questionnaires.

In one embodiment, the NMN, a pharmaceutically acceptable precursor thereof, a pharmaceutically acceptable derivative thereof, or a pharmaceutically acceptable salt thereof, and the composition according to the invention are used in order to reduce hyperalgesia. In the context of the invention, hyperalgesia is defined as an exacerbated pain in response to a pain inducing stimulus.

In one embodiment, the pain is a visceral pain. In the context of the invention, visceral pain results from the activation of nociceptors of the thoracic, abdominal and pelvic organs. Such pains may include, in particular, the sensation of distension, cuts, burns, and combinations thereof. ‘Distension’ refers to any symptom such as stretching, spasm, pulling, pressure or squeezing, and twisting. Visceral pain may be caused in particular by sudden traction on the mesentery, stretching of a serosa, compression of a viscus causing secondary distension, distension of a hollow organ such as the stomach or intestine. From the clinical perspective, visceral pain may be caused by an inflammation, an infection, a disruption of normal mechanical processes such as gastrointestinal motility disorder, a tumour and an ischemia.

In one embodiment, the visceral pain is a pain that is felt within the female or male urogenital tract. The urogenital (or genitourinary) system includes the kidneys, the ureters, the bladder, the urethra, the female reproductive system, the male reproductive system, the ovaries, and the testicles.

Such pain may in particular be indicative of interstitial cystitis. Interstitial cystitis or painful bladder syndrome is an inflammatory disease of the bladder which is characterised by abnormal urges to urinate (urgent and/or frequent urges) and by significant pain in the lower abdomen and the bladder, with the specific site of the pain being the urethra (tube that conveys the urine from the bladder to the exterior of the body) or the vagina in women, sometimes accompanied by difficulty in urinating. The severity of symptoms varies from person to person. The pain may also be due to an inflammation of the urogenital tract which is not attributable to a bacterial, fungal or viral source. For example, the pain may be caused by non-microbial cystitis.

In one embodiment, the visceral pain is pain caused by a urinary tract infection. The urinary tract infection may be caused by a bacterial-, fungal-, or viral infection, or combinations thereof. The urinary tract infection may affect multiple organs of the urogenital system such as the kidneys, the ureters, the bladder, the urethra, and the prostate. It is often manifested in the form of a pain with or without a burning sensation during urination. The urinary tract infection may be cystitis, urethritis, pyelonephritis, prostatitis, or combinations thereof.

Cystitis is the most common form of urinary tract infection. It is an inflammation of the bladder. Cystitis may be caused by the proliferation of intestinal bacteria such as Escherichia coli, large numbers of which are found around the anus. In women, the bacteria pass from the anal region to the bladder via the urinary meatus, and thereby cause local inflammation. In men, cystitis is often caused by a bacterial infection such as chlamydia or gonococcal infections. Cystitis may sometimes be caused by fungal infection with a fungus such as Candida albicans.

Urethritis refers to an inflammation of the urethra, that is to say the tube that connects the bladder to the urinary meatus. Given that in males the prostate is situated near the urethra, the inflammation could also affect the prostate, in which case the condition would correspond to prostatitis.

Pyelonephritis refers to the inflammation of the renal pelvis, that is to say the cavity that collects the urine in the kidney. It is often caused by a bacterial infection, and in particular by poorly treated cystitis. In this case, the bacteria travel up along the ureter so as to infect the kidney.

In one preferred embodiment, the NMN, a pharmaceutically acceptable precursor thereof, a pharmaceutically acceptable derivative thereof, or a pharmaceutically acceptable salt thereof, and the composition according to the invention are used for treating or preventing pain caused by cystitis. Thus, the NMN, a pharmaceutically acceptable precursor thereof, a pharmaceutically acceptable derivative thereof, or a pharmaceutically acceptable salt thereof, and the composition according to the invention provide the means to treat the pain caused by urinary tract infections.

### Mode of Administration and Galenic Form

The NMN, a pharmaceutically acceptable precursor thereof, a pharmaceutically acceptable derivative thereof, or a pharmaceutically acceptable salt thereof, and the composition according to the invention are administered via various routes: oral, ocular, inhalation, sublingual, intravenous, intraarterial, intramuscular, subcutaneous, transcutaneous, vaginal, epidural, topical, intravesical, or rectal. Preferably, the NMN and the composition according to the invention are administered via the oral route.

The composition according to the invention may be in the form of a tablet, a capsule, a sachet, a granule, a soft capsule, a lyophilisate, a lozenge, a suspension, a gel, a syrup, a solution, a water/oil emulsion, an oil/water emulsion, an oil, a cream, a milk, a spray, an ointment, an ampoule, a suppository, an eye drop, a vaginal ovule, a vaginal capsule, a liquid for inhalation, a dry powder inhaler, a pressurised metered dose inhaler. Preferably, the composition according to the invention is in the form of a gastro-resistant capsule or a sublingual tablet.

The term “gastro-resistant” is understood to refer to a galenic form which does not dissolve in the stomach. Such galenic forms are designed with delayed-release, that is to say they have a coating or a coating composition that is resistant to the acid pH of the stomach (pH<2) so as to be able to dissolve in the intestine. The gastro-resistant nature is determined by following the test established by the European Pharmacopoeia. Briefly put, the gastro-resistant nature of a capsule is measured in 0.1 M hydrochloric acid at 37° C. as the disintegration medium in a disintegration apparatus. This medium mimics the physicochemical conditions of the stomach. The capsules are incubated in this medium for a period of 1 hour. The capsule should show no signs of disintegration or cracks which could lead to the loss of contents therein. The capsule is then incubated for a period of 1 hour in a phosphate buffer solution having pH 6.8 and at 37° C., with this solution mimicking the conditions of the intestinal environment in accordance with the recommendations of the European Pharmacopoeia. The capsule should be completely disintegrated in less than one hour.

The term “sublingual tablet” is understood to mean a galenic form that is to be placed under the tongue in order for the active ingredient to be absorbed by the sublingual mucosa, and in particular by the ranine veins and arteries.

The composition according to the invention may also be in a galenic form designed with immediate-release: such a galenic form serves to enable rapid absorption of the nicotinamide adenine dinucleotide (NAD) precursor and thus a reduced onset of action. The immediate-release galenic forms include in particular dispersible tablets, orodispersible tablets, effervescent tablets, and oral lyophilisates.

The composition according to the invention may also be in a galenic form designed with slow (retarded) release. The dissolution and absorption of the NAD precursor take place within the intestine, which thereby limits gastric irritation or the degradation of active ingredients that are fragile in acidic pH. These are mainly gastro-resistant forms, that is to say that the tablets or granules are coated with a polymer film, which is insoluble in acid media but permeable to water in alkaline media or lipid media degraded by intestinal lipases.

The composition according to the invention may also be in a galenic form designed with sequential and sustained release. The galenic forms with sequential release (release at precise time interval) and sustained release (continuous release of the active ingredient until depletion) promote the release of the active ingredient spread out over time in order to ensure an effective plasma concentration is maintained over a longer period in the body of the patient. Such galenic forms provide the means to obtain relief from pain for the patient over a longer period of time, and to space out the doses of medication.

The appropriate mode of administration and the galenic form are determined by the person skilled in the art on the basis of the anatomical location of the pain to be treated and relevant information on the patient. In this regard reference is made to the latest edition of Remington's Pharmaceutical Sciences.

The compositions according to the invention may be formulated with support substances or carriers, excipients and diluents that are suitable per se for these formulations, such as lactose, dextrose, sucrose, sorbitol, mannitol, starches, gum acacia, calcium phosphate, alginates, gum tragacanth, gelatin, calcium silicate, microcrystalline cellulose, polyvinylpyrrolidone, polyethylene glycol, cellulose, water (sterile), methylcellulose, methyl and propyl hydroxybenzoates, talc, magnesium stearate, edible oils, plant and mineral oils, or suitable mixtures thereof. The formulations may optionally contain other substances that are commonly used in pharmaceutical formulations, such as lubricating agents, wetting agents, emulsifying agents and suspending agents, dispersing agents, disintegrants, bulking agents, fillers, preservatives, sweeteners, flavouring agents, flow regulators, mould release agents, etc. The compositions may also be formulated in such a manner as to enable rapid, sustained or slow (retarded) release of the active compound or compounds that are contained therein.

The compositions according to the invention are preferably in the form of unit doses and may be packaged in an appropriate manner, for example in a box, a blister pack, a vial, a bottle, a sachet, an ampoule, or in any other suitable support or container appropriate for single-dose or multi-dose packaging (which may be properly labelled); optionally with one or more package leaflet(s) containing relevant information on the product and/or instructions for use.

### Therapeutic Combinations

Advantageously, the NMN, a pharmaceutically acceptable precursor thereof, a pharmaceutically acceptable derivative thereof, or a pharmaceutically acceptable salt thereof, and the composition according to the invention are used in combination with at least one additional therapeutic agent. In one embodiment, the composition according to the invention comprises the NMN, a pharmaceutically acceptable precursor thereof, a pharmaceutically acceptable derivative thereof, or a pharmaceutically acceptable salt thereof, a pharmaceutically acceptable excipient, and at least one additional therapeutic agent.

Advantageously, the at least one additional therapeutic agent is selected from among antibiotics, antifungals, antivirals, and combinations thereof. Thus, the use of NMN, a pharmaceutically acceptable precursor thereof, a pharmaceutically acceptable derivative thereof, or a pharmaceutically acceptable salt thereof, or of the composition according to the invention makes it possible to reduce the nociceptive pain linked to a bacterial-, fungal-, or viral infection, or combinations thereof. For example, the NMN, a pharmaceutically acceptable precursor thereof, a pharmaceutically acceptable derivative thereof, or a pharmaceutically acceptable salt thereof, or the composition according to the invention may be used in combination with an antibiotic. Such combinations may prove to have utility for topical applications, for example for lozenges for the throat that make it possible on the one hand to reduce the microbial load in the event of bacterial angina and to relieve the pain associated with local inflammation. The NMN, a pharmaceutically acceptable precursor thereof, a pharmaceutically acceptable derivative thereof, or a pharmaceutically acceptable salt thereof, or the composition according to the invention may also be used in combination with an antifungal. Such combinations, for example, may prove to be useful for treating or preventing urinary tract-, vaginal-, cutaneous mycoses, and the like.

In one embodiment, the antibiotic may be selected from among beta-lactams, cyclins, macrolides, related macrolides, quinolones, fluoroquinolones, quinolines, aminoglycosides, fusidic acids, lincosamides, phenicols, polymyxins, sulphonamides, whether or not associated with trimethoprim, antilepers, fosfomycin, mupirocin, nitrofurantoin, nitrofurans, nitroimidazoles, oxazolidinones, glycopeptides, lipopeptides, polymixins, and antituberculosis drugs.

In one embodiment, the antifungal may be selected from among amphotericin B, flucytosine, azole derivatives, terbinafine, selenium sulphide, trichlocarban, nystatin, griseofulvin, amorolfine, salicylic acid, ciclopiroxolamine, amorolfine, tolfanate, and combinations thereof.

The antifungal azole derivatives may in particular be selected from among bifonazole, clotrimazole, econazole, fenticonazole, fluconazole, isoconazole, itroconazole, ketoconazole, miconazole, omoconazole, oxiconazole, sertaconazole, sulconazole, tioconazole, voriconazole, and combinations thereof.

The antivirals may in particular be selected from among antagonists of CCR5 receptor (C—C chemokine receptor type 5), systemic antivirals, phosphoric acid derivatives, integrase inhibitors, fusion inhibitors, neuraminidase inhibitors, non-nucleoside reverse transcriptase inhibitors, nucleoside reverse transcriptase inhibitors, protease inhibitors, nucleosides and nucleotides, excluding reverse transcriptase inhibitors, and combinations thereof.

In one embodiment, the composition according to the invention also comprises at least one analgesic. The analgesic substance may belong to level I, level II, level Ill analgesics according to the WHO classification, or combinations thereof.

In one embodiment, the level I analgesic is selected from among paracetamol, aspirin, non-steroidal anti-inflammatories, cortisone derivatives, and combinations thereof.

The non-steroidal anti-inflammatory may be selected from among ibuprofen, ketoprofen, naproxen, alminoprofen, aceclofenac, mefenamic acid, niflumic acid, tiaprofenic acid, celecoxib, dexketoprofen, diclofenac, etodolac, etoricoxib, fenoprofen, flurbiprofen, indomethacin, meloxicam, nabumetone, piroxicam, sulindac, tenoxicam, and combinations thereof.

The cortisone derivative may be selected from among betamethasone, ciprofloxacin, cortivazol, dexamethasone, fludrocortisone, methylprednisolone, prednisolone, and triamcinolone

The level II analgesic may be selected from among codeine, dihydrocodeine, tramadol, and combinations thereof.

The level Ill analgesic may be selected from among morphine, buprenorphine, fentanyl, hydromorphone, nalbuphine, oxycodone, pethidine, and combinations thereof.

In the context of the present invention, an “excipient” refers to any substance other than the NMN that is in the composition and has no therapeutic effect. The excipient does not interact chemically with the NMN or any other additional therapeutic agent.

The excipient may be selected from among a bulking agent, a lubricant, a flavouring agent, a colouring agent, an emulsifier, a compression agent, a diluent, a preservative, a gelling agent, a plasticiser, a surfactant, or combinations thereof. A person skilled in the art would know to determine the excipient to be selected based on the galenic form that they would have selected.

The composition according to the invention may be a pharmaceutical composition. In this case, the excipient is a pharmaceutically acceptable excipient as defined here above.

The composition according to the invention may also be a dietary supplement.

### Derivatives and Precursors of NMN

According to the invention, the NMN derivative may be selected from among alpha nicotinamide mononucleotide (α-NMN), dihydronicotinamide mononucleotide (denoted as NMN—H), the compound having the formula (I):

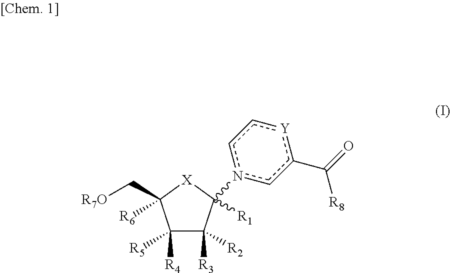

or a stereoisomer thereof, a salt thereof, a hydrate thereof, a solvate thereof, or a pharmaceutically acceptable crystal thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄, and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R₉R₁₀, and P(S)R₉R₁₀; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO₂; wherein each of R₁₁ₐ is independently selected from H and a (C₁-C₆) alkyl, and R_{11b} is independently selected from OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₆-C₁₀) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
   R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are selected independently of one another, from among H, (C₁-C₈) alkyl, (C₁-C₈) alkyl aryl, and —CR_{B}R_{C}—C(O)—OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃;
[symbol] represents a single or a double bond along Y; and
[symbol] represents the alpha or beta anomer depending on the position of R₁;
or a stereoisomer thereof, a salt thereof, a hydrate thereof, a solvate thereof, or a crystal thereof;
or
the compound having the formula (II):

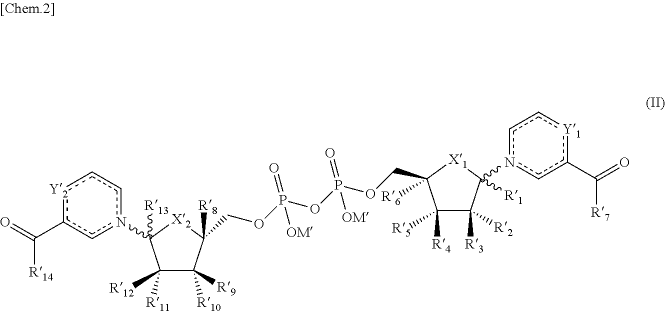

or a stereoisomer thereof, a salt thereof, a hydrate thereof, a solvate thereof, or a crystal thereof; in which
X′₁ and X′₂ are independently selected from among O, CH₂, S, Se, CHF, CF₂, and C═CH₂;
R′₁ and R′13 are independently selected from among H, azido, cyano, a C1-C8 alkyl, a C1-C8 thio-alkyl, a C1-C8 heteroalkyl, and OR, wherein R is selected from H and a C1-C8 alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and halogen; wherein R and R′ are independently selected from H and un (C₁-C₈) alkyl aryl;
Y′₁ and Y′₂ are independently selected from among CH, CH₂, C(CH₃)₂, or CCH₃;
M′ is selected from H or a suitable counter ion;
[symbol] represents a single or double bond depending on Y′₁ and Y′₂; and
[symbol] represents an alpha or beta anomer depending on the position of R′₁ and R′₁₃;
   and combinations thereof.

In a preferred first embodiment, the pharmaceutically acceptable derivative is the compound having the formula (I).

In one variant of the first embodiment, X represents an oxygen.

In one variant of the first embodiment, R₁ and R₆ each independently of one another represent a hydrogen.

In one variant of the first embodiment, R₂, R₃, R₄ and R₅ each independently of one another represent a hydrogen or an OH.

In one variant of the first embodiment, Y represents a CH.

In one variant of the first embodiment, Y represents a CH₂.

In one variant of the first embodiment, R₇ represents a hydrogen.

In one variant of the first embodiment, R₇ represents P(O)(OH)₂.

In one variant of the first embodiment, the compound of the invention is selected from among the compounds having the formula I-A to I-H:

TABLE 1

| Compound No | |
|---|---|
| (Anomer) | Structure |
| I-A (beta) | 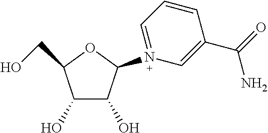 |
| I-B (alpha) | 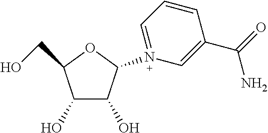 |
| I-C (beta) | 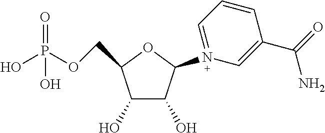 |
| I-D (alpha) | 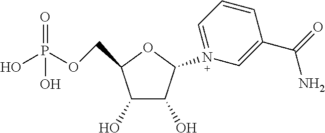 |
| I-E (beta) | 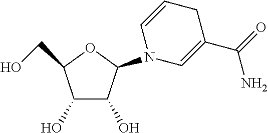 [SMILES: NC(=O)C1=CN([C@@H]2O[C@H](CO)[C@@H](O)[C@H]2O)C=CC1] |
| I-F (alpha) | 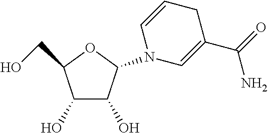 [SMILES: NC(=O)C1=CN([C@H]2O[C@H](CO)[C@@H](O)[C@H]2O)C=CC1] |
| I-G (beta) | 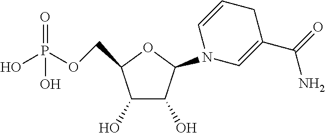 [SMILES: NC(=O)C1=CN([C@@H]2O[C@H](COP(=O)(O)O)[C@@H](O)[C@H]2O)C=CC1] |
| I-H (alpha) | 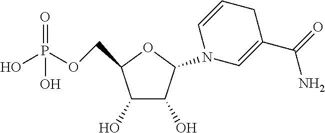 [SMILES: NC(=O)C1=CN([C@H]2O[C@H](COP(=O)(O)O)[C@@H](O)[C@H]2O)C=CC1] |

In one preferred variant of the preferred first embodiment, the pharmaceutically acceptable derivative is alpha-NMN having the formula:

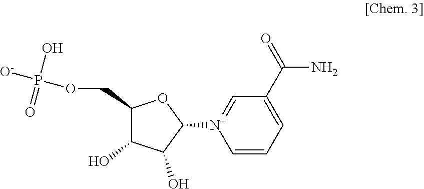 [SMILES: NC(=O)c1ccc[n+]([C@H]2O[C@H](COP(=O)([O-])O)[C@@H](O)[C@H]2O)c1]

In a preferred second embodiment, the pharmaceutically acceptable derivative is the compound having the formula (II).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11, and R′12 each independently represent a hydrogen.

In one variant of the second embodiment, R′2, R′3, R′4, R′5, R′9, R′10, R′11, and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound according to the invention is selected from among the compounds having the formula II-A to II-F:

TABLE 2

| Compound No (Anomer) | Structure |
|---|---|
| II-A (bêta, bêta) | 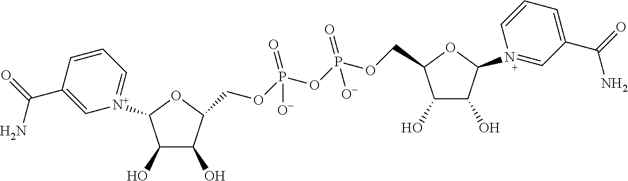 |
| II-B (bêta, alpha) | 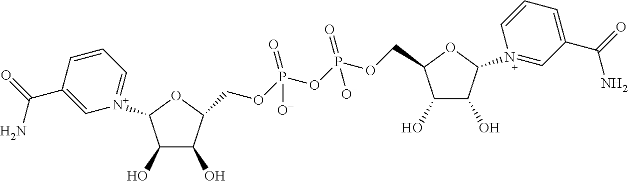 |
| II-C (alpha, alpha) | 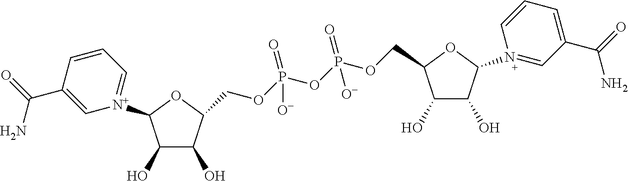 |
| II-D (bêta, bêta) | 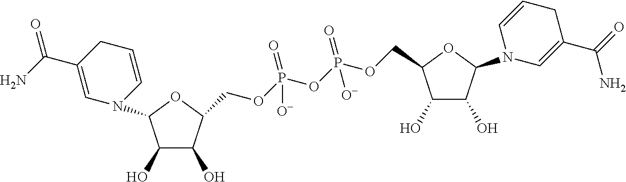 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| II-E (bêta, alpha) | 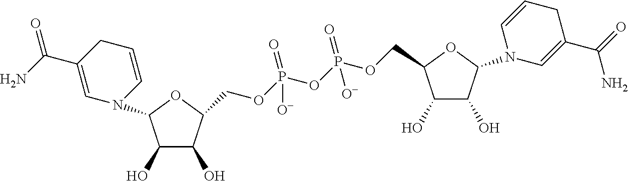 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| II-F (alpha, alpha) | 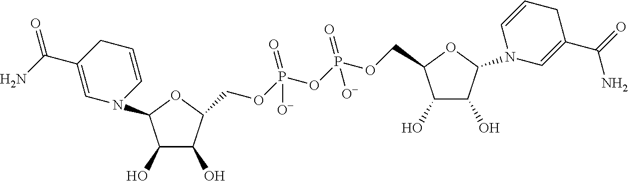 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |

In a preferred fourth embodiment, the pharmaceutically acceptable derivative is NMN—H:

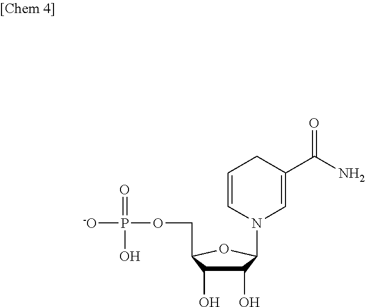

Advantageously, the pharmaceutically acceptable precursor is nicotinamide riboside (denoted NR):

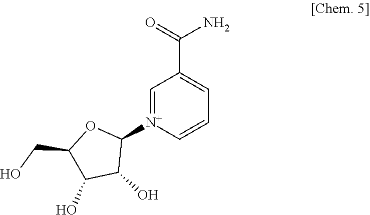 [SMILES: NC(=O)c1ccc[n+]([C@@H]2O[C@H](CO)[C@@H](O)[C@H]2O)c1]

or dihydronicotinamide riboside (denoted —NR—H) having the formula:

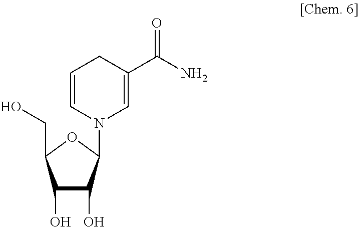

or a combination thereof. Preferably, the precursor is nicotinamide riboside (NR).

Preferably, the NMN derivative is dihydronicotinamide mononucleotide (NMN—H) and/or alpha-NMN.

### Compound Preparation Method for Preparing the Compounds Having the Formula (I) and (II)

The derivatives having the formula (I) or the formula (II) may be prepared according to any method well known to the person skilled in the art.

### Compound Preparation Method for Preparing the Compounds Having the Formula (I)

The derivatives having the formula (I) may be prepared according to the method described in international patent application WO 2017/024255A1.

In particular, the derivatives having the formula (I) as well as alpha-NMN may be prepared according to the method described here below.

In particular, the compounds having the formula (I) disclosed herein may be prepared as described here below from the substrates A-E. It is to be understood by the person skilled in the art that these reaction schemes are by no means intended to be limiting and that variations thereto may be made without departing in spirit and scope from the present invention.

According to one embodiment, the invention relates to a compound preparation method for preparing the compounds having the formula (I) as described here above.

The method involves, in a first step, the mono-phosphorylation of a compound having the formula (A), in the presence of phosphoryl chloride and a trialkyl phosphate, so as to thereby yield the phosphorodichloridate having the formula (B),

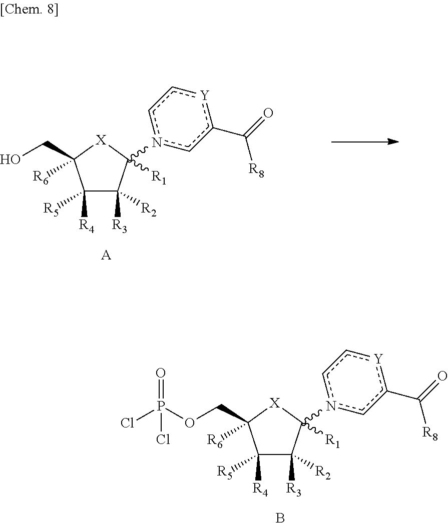

in which X, R₁, R₂, R₃, R₄, R₅, R₆, R₈, Y, [symbol] and [symbol] are as defined here above for the compounds having the formula (I).

In a second step, the phosphorodichloridate having the formula (B) is hydrolysed so as to thereby yield the phosphate having the formula (C),

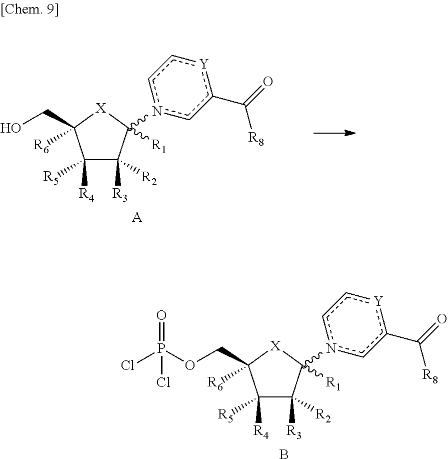

in which X, R₁, R₂, R₃, R₄, R₅, R₆, R₈, Y, [symbol] and [symbol] are as defined here above for the compounds having the formula (I).

According to one embodiment, the compound having the formula (A) is synthesised by means of various methods known to the person skilled in the art.

According to one embodiment, the compound having the formula (A) is synthesised by reaction of the pentose having the formula (D) with a nitrogenous derivative having the formula (E), in which R, R₂, R₃, R₄, R₅, R₆, R₇, Y, are as described here above for the compounds having the formula I, so as to thereby yield the compound having the formula (A-1) which is then selectively deprotected in order to give the compound having the formula (A),

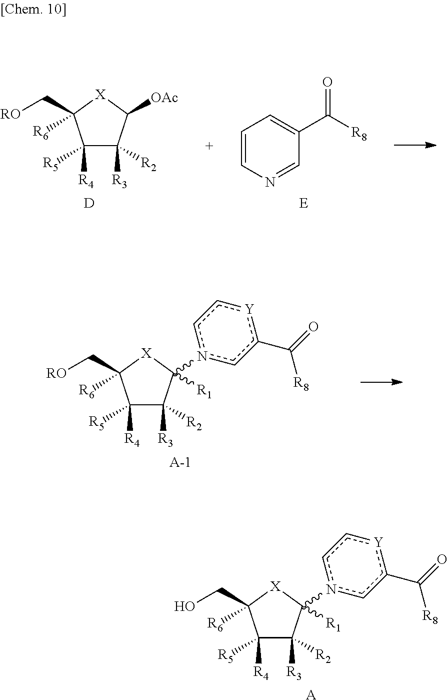

in which X, R₁, R₂, R₃, R₄, R₅, R₆, R₈, Y, [symbol] and [symbol] are as defined here above for the compounds having the formula (I).

According to one embodiment, R is a suitable protecting group known to the person skilled in the art. In one embodiment, the protecting group is selected from among triarylmethyls and/or silyls. Without limitation, some examples of triarylmethyl include trityl, monomethoxytrityl, 4,4′-dimethoxytrityl, and 4,4′,4″-trimethoxytrityl groups. Without limitation, some examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl

According to one embodiment, any hydroxyl group attached to the pentose is protected by an appropriate protecting group known to the person skilled in the art.

The selection and exchanging of the protecting groups is well within the scope of knowledge and expertise of the person skilled in the art. The protecting groups may also be removed by methods well known to the person skilled in the art, for example, with an acid (for example, an inorganic or organic acid), a base or a fluoride source.

In one preferred embodiment, the nitrogenous derivative having the formula (E) is coupled to the pentose having the formula (D) by a reaction in the presence of a Lewis acid so as to thereby yield the compound having the formula (A-1). Without limitation, some examples of Lewis acids include Trimethylsilyl Trifluoromethanesulfonate (TMSOTf), BF₃·OEt₂, TiCl₄ and FeCl₃.

In one embodiment, the method of the present invention additionally also comprises a reduction step of reducing the compound having the formula (A) by various methods well known to the person skilled in the art, so as to thereby yield the compound having the formula (A′) in which is CH₂, and R₁, R₂, R₃, R₄, R₅, R₆, R₈, Y, [symbol] and [symbol] are as defined here above for the compounds having the formula (I).

In one particular embodiment, the present invention relates to a compound preparation method for preparing the compounds having the formula I-A, I-C, I-E, I-G.

In a first step, the nicotinamide having the formula E is coupled to the ribose tetraacetate having the formula D by a coupling reaction in the presence of a Lewis acid, so as to thereby yield the compound having the formula A-1:

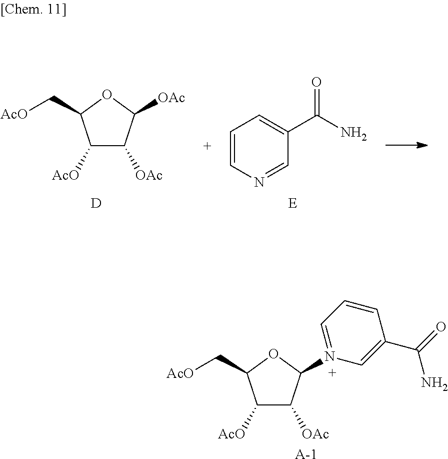

In a second step, an ammoniacal treatment of the compound having the formula A-1 is carried out, so as to thereby yield the compound having the formula I-A:

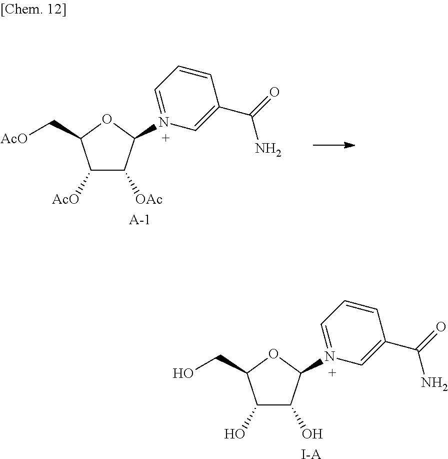

In a third step, the mono-phosphorylation of the compound having the formula I-A, in the presence of phosphoryl chloride and a trialkyl phosphate, thereby yields the phosphorodichloridate having the formula I-A:

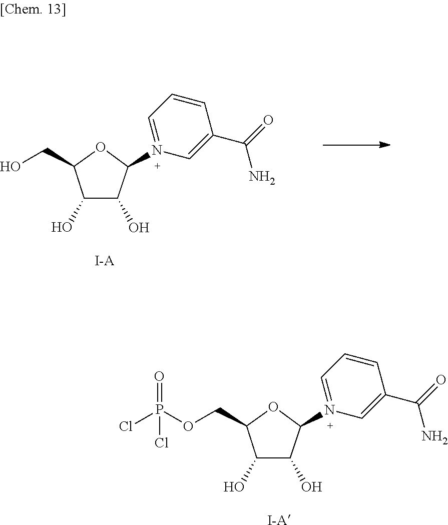

In a fourth step, the phosphorodichloridate having the formula B is hydrolysed so as to thereby yield the compound having the formula I-C:

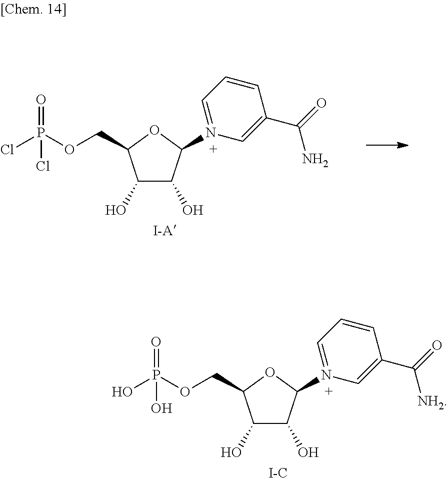

In one embodiment, a reduction step of reducing the compound having the formula I-A is carried out, so as to thereby yield the compound having the formula I-E.

The compound having the formula I-E is then mono-phosphorylated as described in the fourth step and hydrolysed so as to thereby yield the compound having the formula I-G.

According to one embodiment, the compounds having the formula (I) are selected from compounds I-A to I-H in the table below:

TABLE 1

| Compound No | |
|---|---|
| (Anomer) | Structure |
| I-A (beta) | 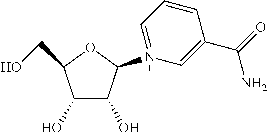 |
| I-B (alpha) | 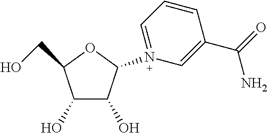 |
| I-C (beta) | 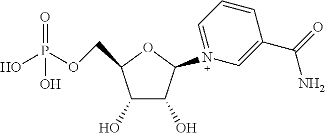 |
| I-D (alpha) | 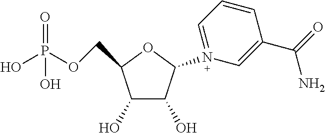 |
| I-E (beta) | 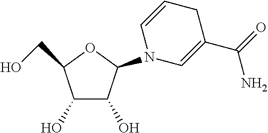 [SMILES: NC(=O)C1=CN([C@@H]2O[C@H](CO)[C@@H](O)[C@H]2O)C=CC1] |
| I-F (alpha) | 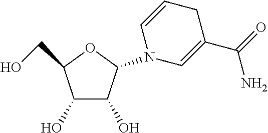 [SMILES: NC(=O)C1=CN([C@H]2O[C@H](CO)[C@@H](O)[C@H]2O)C=CC1] |
| I-G (beta) | 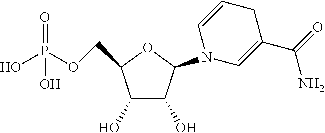 [SMILES: NC(=O)C1=CN([C@@H]2O[C@H](COP(=O)(O)O)[C@@H](O)[C@H]2O)C=CC1] |
| I-H (alpha) | 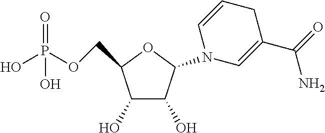 [SMILES: NC(=O)C1=CN([C@H]2O[C@H](COP(=O)(O)O)[C@@H](O)[C@H]2O)C=CC1] |

In one preferred embodiment, the compounds of the invention are the compounds having the formula I-A, I-C, I-E, and I-G as in the table above, or a pharmaceutically acceptable salt and/or solvate thereof. In an even more preferred embodiment, the compound is the compound having the formula I-C or I-D, a pharmaceutically acceptable salt and/or solvate thereof.

### Derivative Preparation Method for Preparing the Derivatives Having the Formula (II)

In particular, the compounds having the formula II presented herein may be prepared as described here below from the substrates X-XIII. It is to be understood by the average person skilled in the art that these diagrams are by no means intended to be limiting and that variations thereto in terms of the detail may be made without departing in spirit and scope from the present invention.

According to one embodiment, the invention relates to a compound preparation method for preparing the compound having the formula I described here above.

The method consists first of all in mono-phosphorylating a compound having the formula X, in the presence of phosphoryl chloride in a trialkyl phosphate, in order to obtain the compound phosphorodichloridate XI,

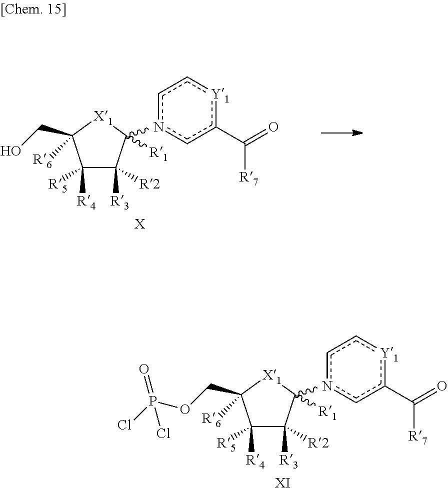

in which X′₁, R′₁, R′₂, R′₃, R′₄, R′₅, R′₆, R′₇, Y′₁, [symbol] and [symbol] are as defined here above.

In a second step, the hydrolysis of the phosphorodichloridate XI obtained in the first step gives the phosphate compound having the formula XII,

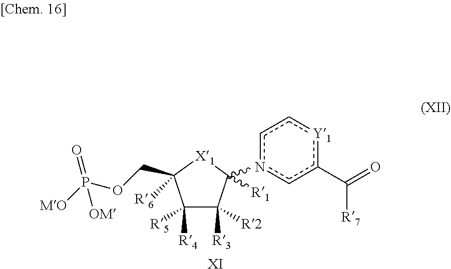

in which X′₁, R′₁, R′₂, R′₃, R′₄, R′₅, R′₆, R′₇, Y′₁, M′, [symbol] and [symbol] as defined here above.

The phosphate compound having the formula XII obtained in the second step is then reacted with a phosphorodichloridate compound having the formula XIII obtained as described in the first step,

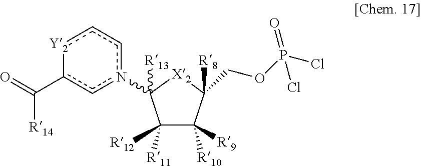

in which X′₂, R′₈, R′₅, R₁₀, R′₁₁, R′₁₂, R′₁₃, R′₁₄, Y′₂, [symbol] and [symbol] are as described herein for formula II, in order to give the compound having the formula II as described herein.

According to one embodiment, the method further comprises a reduction step of reducing the compound having the formula II, using various methods known to specialists, in order to give the compound having the formula II, where Y′₁ and Y′₂ are identical and each represent CH₂, and where X′₁, X′₂, R′₁, R′₂, R′₃, R′₄, R′₅, R′₆, R′₇, R′₈, R′₉, R₁₀, R′₁₁, R′₁₂, R′₁₃, R′₁₄, Y′₁, Y′₂, and [symbol] , [symbol] are as described herein for formula II.

In one variant, R is a suitable protecting group known to the person skilled in the art. Triarylmethyl and/or silyl groups are examples of suitable protecting groups. Without limitation, some examples of triarylmethyl include trityl, monomethoxytrityl, 4,4′-dimethoxytrityl, and 4,4′,4″-trimethoxytrityl. Without limitation, some examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl.

According to one representation, any hydroxy group attached to the pentose ring is protected by a suitable protecting group known to the person skilled in the art.

The selection and exchanging of the protecting groups is well within the scope of knowledge and expertise of the person skilled in the art. Any protecting group may also be removed by methods known in the art, for example, with an acid (for example, an inorganic or organic acid), a base or a fluoride source.

According to one preferred embodiment, the nitrogen compounds having the formula XV are added to the pentose XIV by a coupling reaction in the presence of a Lewis acid in order to give the compound having the formula X-1. Without limitation, some examples of suitable Lewis acids include Trimethylsilyl Trifluoromethanesulfonate (TMSOTf), BF₃·OEt₂, TiCl₄ and FeCl₃.

According to one specific embodiment, the invention relates to a compound preparation method for preparing the compound having the formula VIII,

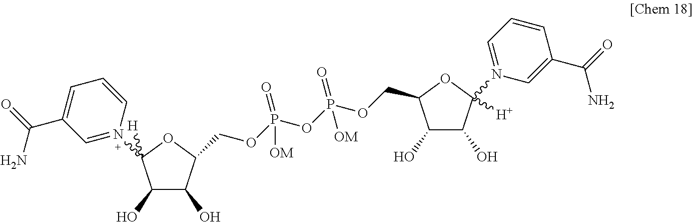

or the pharmaceutically acceptable salts and/or solvates thereof.

In a first step, the nicotinamide having the formula XV is added to the ribose tetraacetate XIV, by a coupling reaction in the presence of a Lewis acid, in order to give the compound having the formula X-1:

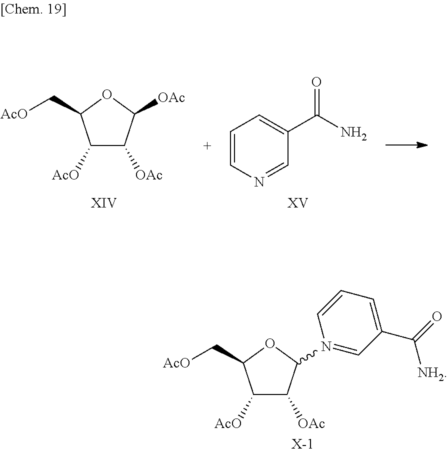

In a second step, an ammoniacal treatment of the compound having the formula X-1 gives the compound having the formula X:

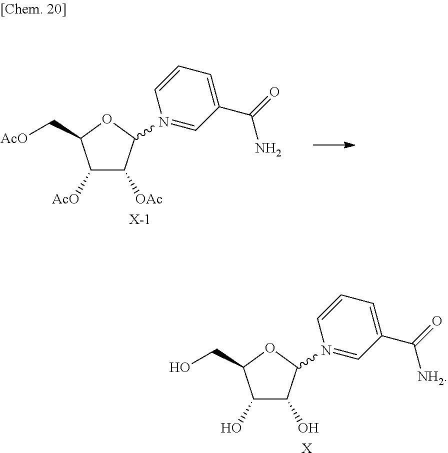

In a third step, the mono-phosphorylation of a compound having the formula X, in the presence of phosphoryl chloride in a trialkyl phosphate, gives the compound phosphorodichloridate XI:

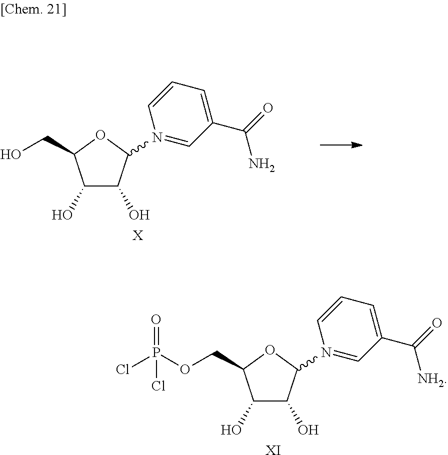

In a fourth step, the phosphorodichloridate compound XI obtained in the third step is partially hydrolysed in order to give the phosphate compound having the formula XII:

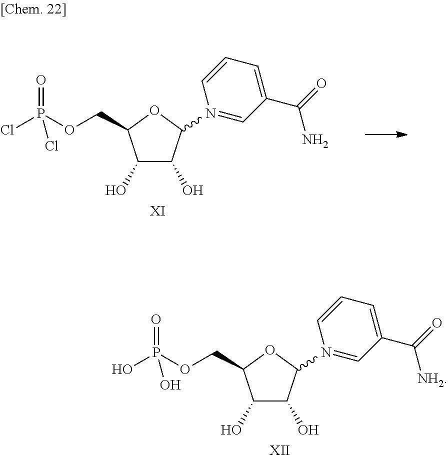

In a fifth step, the phosphate compound having the formula XII obtained in the fourth step is then reacted with the phosphorodichloridate compound having the formula XI obtained as described in the third step, in order to obtain the compound having the formula VIII.

According to another specific implementation embodiment, the invention relates to a compound preparation method for preparing the compound having the formula IX,

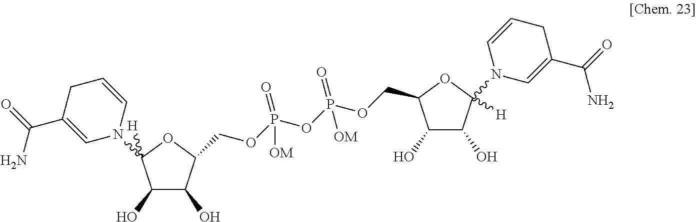

or the pharmaceutically acceptable salts and/or solvates thereof.

According to one variant, the compound having the formula IX is obtained from the compound having the formula VIII, which is synthesised beforehand as described here above.

In this embodiment, the compound having the formula IX is obtained by reducing the compound having the formula VIII, using a suitable reducing agent known to the specialised person skilled in the art, in order to give the compound having the formula IX.

According to one embodiment, the preferred compounds of the invention are the compounds II-A to II-F, listed in Table 2:

TABLE 2

| Compound No (Anomer) | Structure |
|---|---|
| II-A (bêta, bêta) | 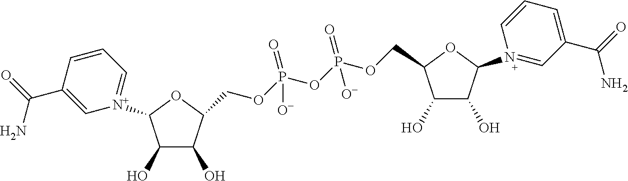 |
| II-B (bêta, alpha) | 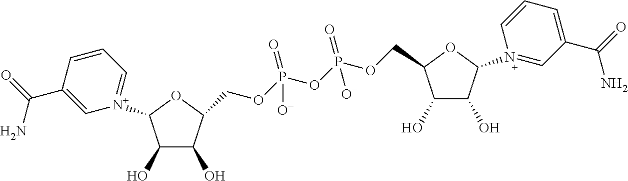 |
| II-C (alpha, alpha) | 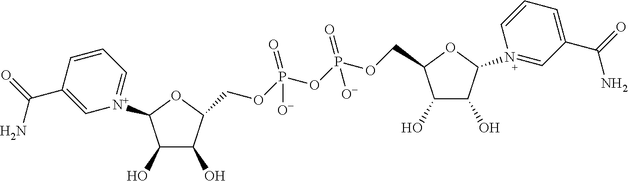 |
| II-D (bêta, bêta) | 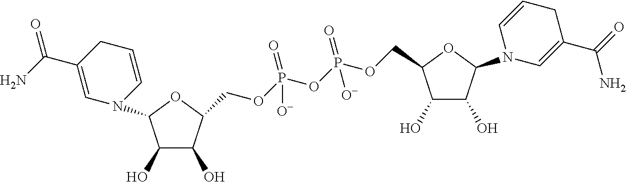 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| II-E (bêta, alpha) | 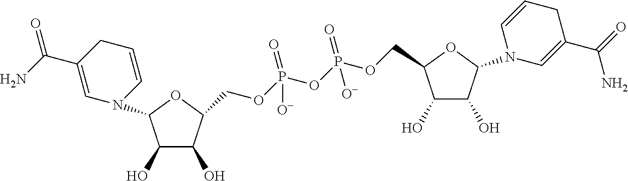 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| II-F (alpha, alpha) | 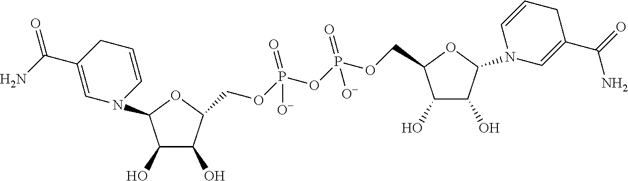 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |

### EXAMPLES

In the remainder of this description, the examples provided are intended by way of illustration of the present invention and are in no way intended to limit the scope thereof.

### Example 1

The effectiveness of the use of NMN according to the invention was evaluated in rats in a model of nociceptive pain. More precisely, the model used is a visceral pain model. The administration of cyclophosphamide (CYP) serves as the means to simulate cystitis in rats.

The positive control is ibuprofen, a non-steroidal anti-inflammatory drug frequently prescribed to relieve nociceptive pain. The negative control is the carrier for NMN and ibuprofen, that is to say, distilled water.

For this study, 7-week-old female Sprague-Dawley rats were divided into three groups, each group comprising of 6 rats:
a control group who received 5 ml of distilled water (carrier);
a group treated with NMN at 500 mg/kg; and
a group treated with ibuprofen at 300 mg/kg.

The NMN is in the form of zwitterion.

After 24 hours of adaptation, a test to measure the threshold of pain tolerance of each animal was carried out with von Frey filaments prior to the start of the study. This measurement prior to any exposure to a painful stimulus makes it possible to obtain the baseline pain tolerance threshold level in the animals and serves as a negative control.

Von Frey filaments are used as a device for measuring the sensitivity of the skin to touch. The use of von Frey filaments makes it possible to test for allodynia and hyperalgesia in rodents. Briefly put, each filament corresponds to a given force. The filaments are applied in ascending order of increasing force to the animal's skin. The rodents in effect have a reflex to withdraw when an unexpected contact occurs. The withdrawal in response to a force exerted on the rodent is indicative of the animal's threshold of tolerance to pain. The use of von Frey filaments is commonly implemented for measurement of pain in rodents (Deuis J R, Dvorakova L S and Vetter I (2017) Methods Used to Evaluate Pain Behaviors in Rodents. Front. Mol. Neurosci. 10:284.).

In the present study, eight filaments in ascending order of increasing force, of 1 g, 2 g, 4 g, 6 g, 8 g, 10 g, 15 g, and 26 g, were used and applied three times each to the animal's abdomen, near the bladder. The animal's response was assessed as follows:
score 0: no response
score 1: retraction of the abdomen
score 2: stamping or changing of position of the animal
score 3: twitching, or curving or rounding of the abdomen, or licking of the area stimulated by the von Frey filament.

For each rat, the results are expressed as follows:
nociceptive threshold: first force level at which a response from the animal is observed (response score greater than or equal to 1). This indicates the lowest threshold of tolerance to pain for measuring allodynia;
nociceptive score: percentage of the maximum response for each filament. This indicates the overall pain response.

The animals of each of the groups receive either the carrier, or NMN at 500 mg/kg, or ibuprofen at 300 mg/kg administered via the oral route. After administration of the compounds to be tested (carrier, NMN, or ibuprofen), cyclophosphamide is injected via the intraperitoneal route into each rat. The cyclophosphamide induces strong inflammation in the bladder and simulates a pain induced by a urinary tract infection such as cystitis.

The test with the von Frey filaments is repeated at 2 hr and then subsequently at 4 hr after injection of the cyclophosphamide in order to measure the pain response of the animals.

The results were analysed by a one-way analysis of variance (ie one-way ANOVA) test supplemented by a Dunnett's test or by a two-way ANOVA analysis. With regard to statistical significance, * signifies that p<0.05, ** signifies that p<0.01, and *** signifies that p<0.001, as compared to the group treated with the carrier.

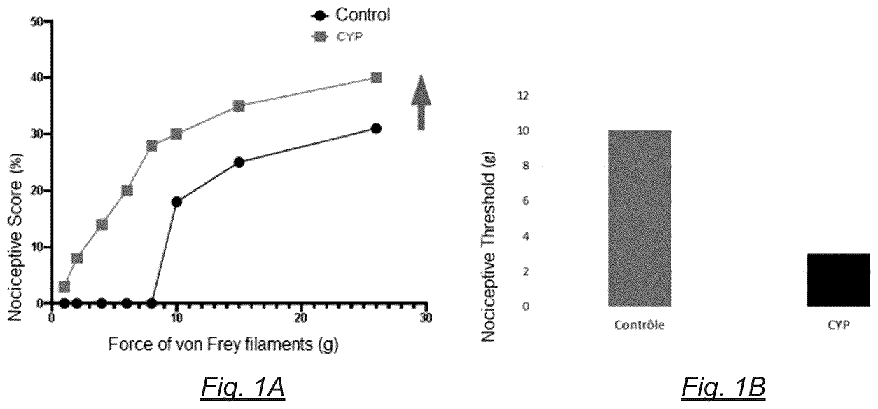

As may be seen in FIG. 1A, the administration of cyclophosphamide elicits a response from the rats immediately upon application of the first filament of 1 g force, while the response from the animals prior to exposure shows that the animals exhibit no response to pain until 10 g prior to administration of cyclophosphamide: the injection of cyclophosphamide therefore lowers the allodynia threshold. This result is corroborated by FIG. 1B which shows that the administration of cyclophosphamide reduces the pain tolerance threshold from 10 g to 3 g. In other words, the allodynia threshold is significantly reduced by the injection of cyclophosphamide in rats. The animals who are treated with cyclophosphamide are therefore more sensitive to pain.

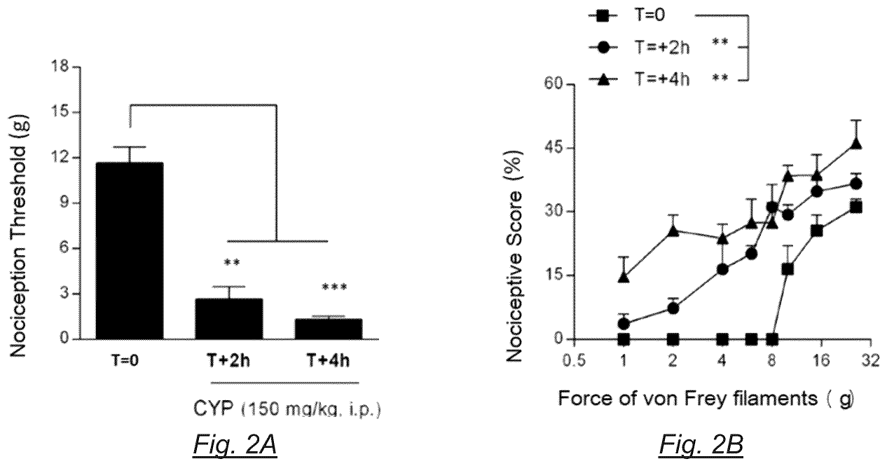

FIGS. 2A and 2B show that the lowering of the allodynia threshold continues, with the animals showing even lower tolerance to pain 4 h after the injection of cyclophosphamide as compared to 2 h after injection.

FIG. 1A also shows that at equal force, the animals feel greater pain: the injection of cyclophosphamide therefore triggers hyperalgesia. FIG. 2B shows that this effect persists over time, with the pain scores measured in the treated rats after 4 hours being higher than those from the measurement carried out 2 hours after the injection.

The injection of cyclophosphamide therefore induces on the one hand a reduction in allodynia and an increase in hyperalgesia in the treated animals, with the effects being more accentuated over time.

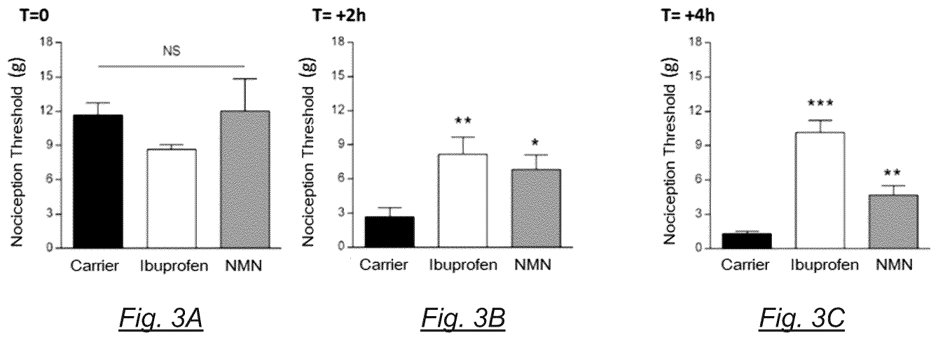

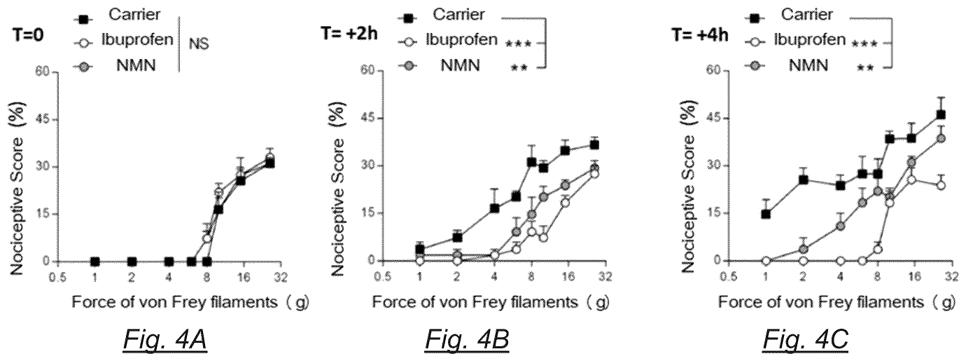

FIGS. 3A, 3B, 3C, 4A, 4B, and 4C show the effects of administration of NMN and ibuprofen on the pain thresholds and scores in the rats treated with cyclophosphamide.

As shown in FIGS. 3A, 3B, and 3C, the administration of NMN provides the ability to increase the response threshold to nociceptive pain significantly, at T0, T=2 h after injection, and T=4 h after injection. In an expected manner, ibuprofen also provides the ability to significantly increase the threshold of tolerance to pain in rats treated with cyclophosphamide at T0, T=2 hours after injection, and T=4 hours after injection. These results show that the administration of NMN provides the ability to reduce allodynia.

As shown in FIG. 4A, the NMN, the ibuprofen, and the carrier show similar curves of nociceptive scores at T0, at the time instant of injection of cyclophosphamide. This demonstrates that the animals do not develop pain in response to the administration of NMN or ibuprofen. On the other hand, FIGS. 4B and 4C show that the administration of NMN serves as the means for reducing the nociceptive score 2 hours and 4 hours after the administration of cyclophosphamide, in the same way as ibuprofen.

The administration of NMN and of a composition comprising the same therefore makes it possible to reduce allodynia and hyperalgesia to a significant extent, and in a manner similar to ibuprofen.

### Example 2

### Synthesis of the Compound of the Invention

### Materials and Methods

All the reagents were obtained from commercial suppliers and used without any further purification. Thin layer chromatography was carried out on TLC silica gel 60 F254 plastic sheets (0.2 mm layer thickness) from Merck. Purification by column chromatography was carried out on silica gel 60 (70-230 mesh ASTM, Merck). The melting points were determined either on a digital device (Electrothermal IA 8103) and are not corrected, or on a Kofler heating bench of type WME (Wagner & Munz). The ¹H, ¹⁹F, and ¹³C nuclear magnetic resonance (NMR) and infrared (IR) spectra confirmed the structures of all of the compounds. The IR spectra were recorded on a Perkin Elmer Spectrum 100 FT-IR spectrometer; and the NMR spectra were recorded, using CDCl₃, CD₃CN, D₂O or DMSO-d₆ as solvent, on a BRUKER AC 300 or 400 spectrometer at 300 or 400 MHz for the ¹H spectra, 75 or 100 MHz spectra for the ¹³C spectra, and 282 or 377 MHz for the ¹⁹F spectra. The chemical shifts (δ) were expressed in parts per million relative to the signal, indirectly (i) with CHCl₃ (δ 7.27) for ¹H; and (ii) with CDCl₃ (δ 77.2) for ¹³C; and directly (iii) with CFCl₃ (internal standard) (δ 0) for ¹⁹F. The chemical shifts are provided in ppm and the peak multiplicities are denoted as follows: s, singlet; br s, broad singlet; d, doublet; dd, doublet of doublets; t, triplet; q, quartet; quint, quintet; m, multiplet. High-resolution mass spectra (HRMS) were obtained from the “Service central d'analyse de Solaize” (French National Center for Scientific Research—Solaize) and were recorded on a Waters spectrometer using electrospray ionisation time-of-flight (ESI-TOF) mass spectrometry.

### Protocol

### Step 1: Synthesis of the Compound Having the Formula X-1

The compound having the formula XIV (1.0 equiv.) is dissolved in dichloromethane. The nicotinamide having the formula XV (1.50 equiv.) and the TMSOTf (1.55 equiv.) are added at ambient temperature. The reaction mixture is heated under reflux and stirred until completion of the reaction. The mixture is cooled to ambient temperature and filtered. The filtrate is concentrated to dryness so as to give crude nicotinamide riboside tetraacetate having the formula X-1.

### Step 2: Synthesis of the Compound Having the Formula X

The crude NR tetraacetate having the formula X-1 is dissolved in methanol and cooled to −10° C. This is followed by addition of 4.6 M ammonia in methanol (3.0 equivalents) at −10° C. and the mixture is stirred at this temperature until completion of the reaction. Dowex HCR (H⁺) is added until a pH of 6-7 is attained. The reaction mixture is heated to 0° C. and filtered. The resin is washed with a mixture of methanol and acetonitrile. The filtrate is concentrated until it becomes dry. The residue is dissolved in acetonitrile and concentrated to solid content dryness. The residue is dissolved in acetonitrile so as to give a solution of crude nicotinamide riboside triflate having the formula X.

### Step 3: Synthesis of the Compound Having the Formula XI

The solution of crude nicotinamide riboside triflate in acetonitrile is diluted with trimethyl phosphate (10.0 equivalents). The acetonitrile is distilled under vacuum and the mixture is cooled to −10° C. Phosphorus oxychloride (4.0 equiv.) is added at −10° C. and the mixture is stirred at −10° C. until completion of the reaction.

### Step 4 and Step 5: Synthesis of the Compound Having the Formula I-A

The mixture is hydrolysed by adding a 50/50 mixture of acetonitrile and water, followed by the addition of methyl tert-butyl ether (or tert-butyl methyl ether). The mixture is filtered and the solid is dissolved in water. The aqueous solution is neutralised by adding sodium bicarbonate and extracted with dichloromethane. The aqueous layer is concentrated to dryness so as to give a crude mixture of NMN and di-NMN having the formula I-A.

Isolation of di-NMN Having the Formula I-A

The NMN and the di-NMN having the formula I-A are separated by purification on Dowex 50w×8 with elution of water. The fractions containing di-NMN are concentrated to solid content dryness. The residue is purified by column chromatography on silica gel (isopropanol/water gradient). The pure fractions are combined and concentrated. The residue is lyophilised so as to give di-NMN as a beige solid.

### Biological Data

The objective of the present study is to evaluate the effects of oral administration of nicotinamide mononucleotide (NMN), alpha- NMN (compound A) and compound I-A (compound B) at 500 mg/kg on the visceral pain response in the model of acute cystitis induced by cyclophosphamide (CYP) in female Sprague-Dawley rats.

### Materials and Methods

Animals: Sprague-Dawley female rats, 7 weeks old as of their birth

### Pharmacological Treatment:

NMN: 500 mg/kg
alpha- NMN: 500 mg/kg
Compound I-A: 500 mg/kg
Carrier: distilled water
Route of administration: per os [p.o.](by oral administration), 5 ml/kg
Frequency of administration: once on DO, 15 min prior to the intraperitoneal injection (i.p.) of CYP.

Acute Cystitis Induced by CYP: The CYP was injected via intraperitoneal injection at 150 mg/kg in a final volume of 5 ml/kg of saline solution.

### Mechanical Stimulation Using Von Frey Filaments

The rats were placed in individual Plexiglas boxes with a metal wire mesh floor and allowed to adapt to the chamber for a period of at least 30 minutes prior to the commencement of any testing. Eight von Frey filaments with increasing levels of force viz 1, 2, 4, 6, 8, 10, 15, and 26 g were used. Each calibrated filament was applied 3 times to the lower abdominal area, near the bladder.

### Evaluation of Nociceptive Behaviours for Each Application

Score 0=no response
Score 1=retraction of the abdomen
Score 2=stamping or changing of position
Score 3=wheezing or squealing, or abdominal curvature, or licking of the site stimulated with von Frey filaments

For each rat, the results were expressed as follows:
Nociceptive threshold: first von Frey force level for which the stimulus is perceived as being painful (the score ≥1 is obtained)=>lowered threshold=allodynia
Nociceptive score: % of the maximum response (total=9 for 3 combined applications) for each filament=>Overall response to pain

Experimental Groups (6 Rats Per Group):
Group 1: Carrier (5 ml/kg)+CYP
Group 2: NMN (500 mg/kg)+CYP
Group 3: Compound A (500 mg/kg)+CYP
Group 4: Compound B (500 mg/kg)+CYP

### Results

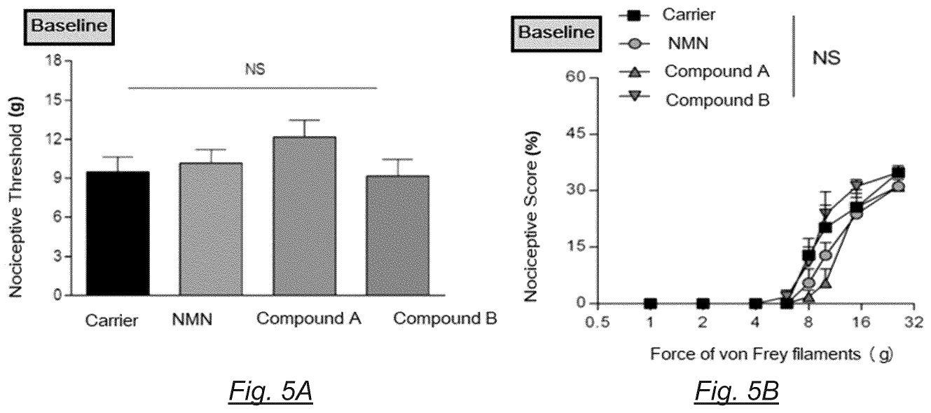

Baseline nociceptive parameters (prior to CYP injection) for all the experimental groups: The results show (FIGS. 5A and 5B) that the baseline nociceptive responses were similar among all of the experimental groups (prior to the injection of CYP).

The visceral pain induced by the CYP 2 hours and 4 hours post injection (as compared to the baseline value in the carrier treated group): The results show that as compared to the baseline response, the CYP (150 mg/kg, i.p.) induced a significant decrease in the nociceptive threshold (FIG. 2A) and a significant increase in the nociceptive scores (FIG. 2B) at the two time points.

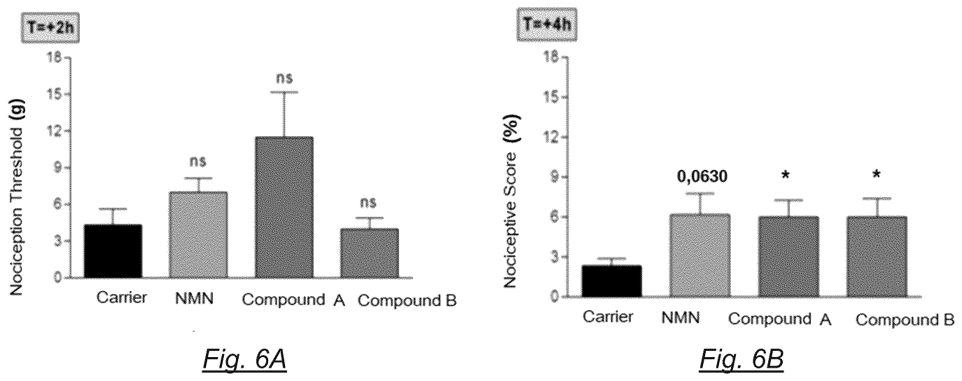

Effects of the NMN, Compound A, and Compound B on the CYP-Induced Allodynia (Nociceptive Threshold): The results show that, as compared to the carrier:
The NMN (500 mg/kg, p.o.) resulted in a slight increase in the nociceptive threshold at +2 hr (FIG. 6A) and +4 hr (FIG. 6B) with an effect just above the margin of statistical significance at +4 hr (p=0.063);
Compound A (500 mg/kg, p.o.) resulted in an increase in the nociceptive threshold at +2 hr (FIG. 6A) and +4 hr (significant at +4 hr) (FIG. 6B);
Compound B (500 mg/kg, p.o.) resulted in a significant increase in the nociceptive threshold only at +4 hr (FIG. 6B) and induced no effect at +2 hr (FIG. 6A).

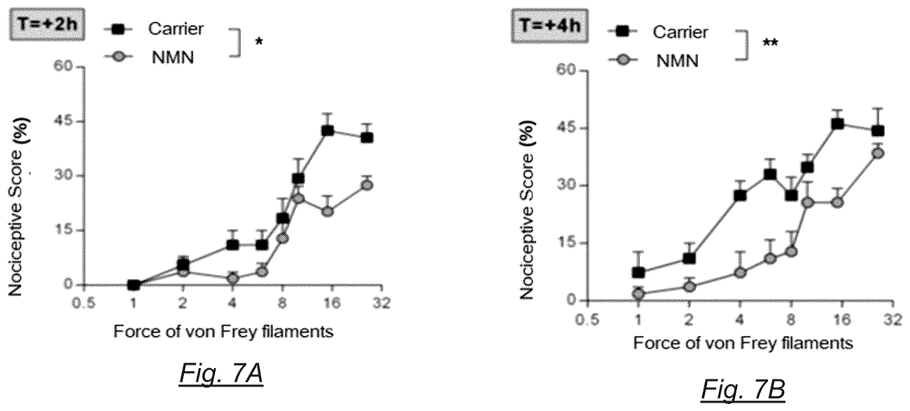

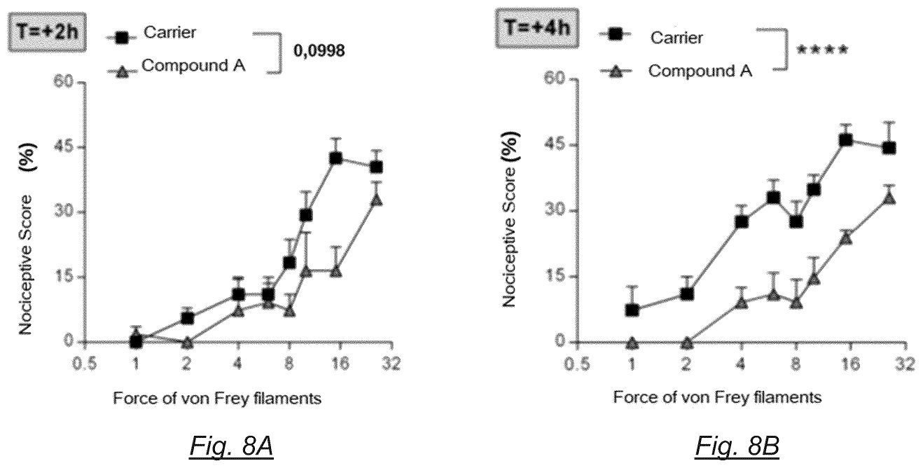

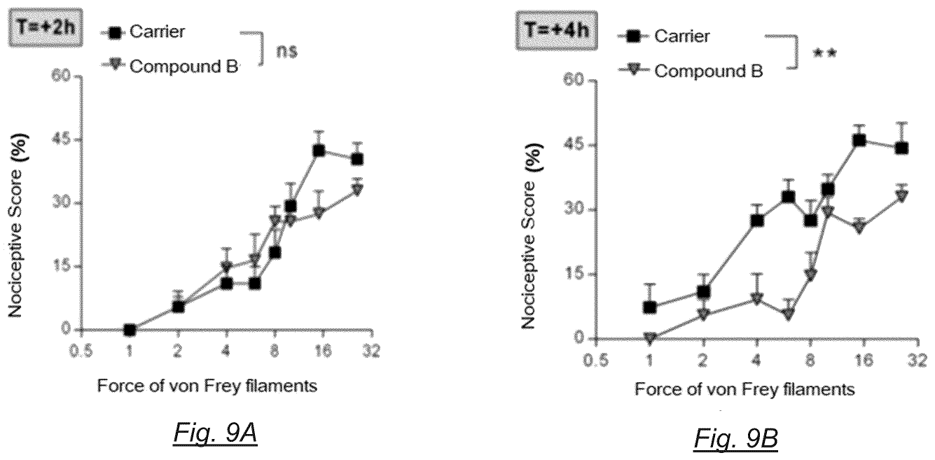

Effects of the NMN, Compound A, and Compound B on the CYP-Induced Visceral Pain (Nociceptive Scores): The results show (FIGS. 7A, 7B, 8A, 8B, 9A, and 9B) that as compared to the carrier:
The NMN (500 mg/kg, p.o.) led to a significant decrease in the nociceptive scores at +2 hr (FIG. 7A) and +4 hr (FIG. 7B);
Compound A (500 mg/kg, p.o.) led to a decrease in the nociceptive scores at +2 hr (FIG. 8A) and +4 hr (FIG. 8B) which only achieved the level of statistical significance at +4 h;
Compound B (500 mg/kg, p.o.) led to a significant decrease in the nociceptive scores at +4 hr (FIG. 9B) (no effect was observed at +2 hr (FIG. 9A)).

### Summary of Results

The baseline nociceptive responses were similar in all of the experimental groups (prior to the injection of CYP).

In comparison with the baseline response, the effects of CYP (150 mg/kg, i.p.) at 2 and 4 hours were characterised by:
A significant decrease in the nociceptive threshold at +2 hours and +4 hours;
A significant increase in the nociceptive scores at +2 hours and +4 hours.

As compared to the carrier, in the rats injected with CYP, the effects of the NMN (500 mg/kg, p.o.) led to:
a slight increase in the nociceptive threshold at +2 hr and +4 hr with an effect just above the margin of statistical significance at +4 hr (p=0.063)
a significant decrease in the nociceptive scores at +2 hr and +4 hr.

As compared to the carrier, in the rats who received an injection of CYP, the effects of Compound A (500 mg/kg, p.o.) were characterised by:
An increase in the nociceptive threshold at +2 hr and +4 hr with an effect which only achieved the level of significance at +4 hr;
A decrease in the nociceptive scores at +2 hr and +4 hr which only achieved the level of statistical significance at +4 hr.

As compared to the carrier, in the rats injected with CYP, the effects of Compound B (500 mg/kg, p.o.) led to:
A significant increase in the nociceptive threshold at +4 hr (no effect was observed at +2 hr);
A significant decrease in the nociceptive scores at +4 hr (no effect was observed at +2 hr).

### CONCLUSION

A single intraperitoneal injection of CYP (150 mg/kg) induced a visceral pain 2 hours and 4 hours after the injection with a more pronounced effect at +4 hours.

A single oral treatment of NMN (500 mg/kg) relieved the visceral pain induced by the CYP at the two time points, with a higher level of significance obtained at +4 hr.

The administration of alpha- NMN (Compound A) reduced the visceral pain induced by the CYP at +2 hr and +4 hr, with its effects however, only achieving the level of significance at +4 hr.

In the rats injected with CYP, the oral treatment with Compound I-A (Compound B) had no beneficial effect at +2 hr but showed significant anti-nociceptive activity at a later time point (that is to say, +4 hr).

The inventors have therefore demonstrated that the use of NMN and the pharmaceutically acceptable derivatives thereof, such as alpha NMN and Compound I-A, as well as the compositions that comprise the same in accordance with the invention, provide the ability to reduce nociceptive pain, and more particularly visceral pain induced by cystitis.

The use of NMN and the pharmaceutically acceptable derivatives thereof, such as alpha NMN and Compound I-A, as well as the compositions that comprise the same in accordance with the invention, therefore provide the ability to treat and prevent pain, in particular nociceptive pain.

What is claimed is:

1. A method for prevention and/or treatment of pain, the pain being visceral nociceptive pain and not neuropathic pain, the method comprising:
administering to a subject a therapeutically effective amount of nicotinamide mononucleotide (NMN), a pharmaceutically acceptable derivative thereof, or a pharmaceutically acceptable salt thereof, wherein the pharmaceutically acceptable derivative is alpha nicotinamide mononucleotide (α-NMN), dihydronicotinamide mononucleotide (denoted as NMN-H), a compound of Formula I: 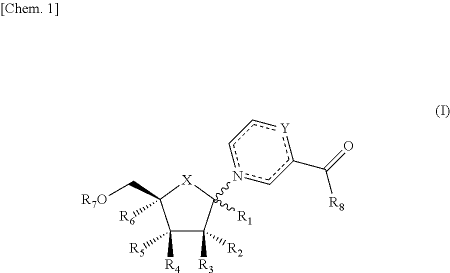
wherein
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄, and R₅ are each independently selected from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R₉R₁₀, and P(S)R₉R₁₀; in which
R₉ and R₁₀ are each independently selected from among OH, OR₁₁, NHR₁₃, NR₁₃R₁₄, a (C₁-C₈) alkyl, a (C₂-C₈) alkenyl, a (C₂-C₈)alkynyl, (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), and —SO₂N(R₁₁ₐ)₂ SO₂; wherein each R₁₁ₐ is independently selected from H and a (C₁-C₆) alkyl, and R_{11b} is independently selected from OH, C₁-C₆ alkoxy, NH₂, NH(C₁-C₆ alkyl) or N(C₁-C₆ alkyl)₂;
R₁₂ is selected from among H, C₁-C₁₀alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from among halogen, trifluoromethyl, C₁-C₆ alkyl, C₁-C₆ alkoxy, and cyano; and
R_{AA} and R_{A′} are each independently selected from among H, a (C₁-C₁₀)alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the aryl groups are optionally substituted with a group selected from among hydroxyl, (C₁-C₁₀) alkyl, (C₆-C₁₀) alkoxy, a halogen, a nitro, and a cyano; or
R₉ and R₁₀ form, together with the phosphorus atoms to which they are attached, a 6-membered ring in which —R₉—R₁₀— represents ₂—CH₂—CHR—; wherein R is selected from among H, a (C₅-C₆) aryl group, and (C₅-C₆) heteroaryl group, wherein the aryl or heteroaryl groups are optionally substituted by a halogen, trifluoromethyl, a (C₁-C₆) alkyl, a (C₁-C₆) alkoxy, and cyano; or
R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the aryl or heteroaryl groups are optionally substituted by a halogen, trifluoromethyl, a (C₁-C₆) alkyl, a (C₁-C₆) alkoxy, or a cyano;
R₈ is selected from among H, OR, NHR₁₃, NR₁₃R₁₄, NH—NHR₁₃, SH, CN, N₃, and halogen; wherein R₁₃ and R₁₄ are each independently selected from among H, (C₁-C₈) alkyl, (C₁-C₈) alkyl aryl, and —CR_{B}R_{C}—C(O) —OR_{D} in which R_{B} and R_{C} are each independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy are each optionally and independently substituted by one or more of halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃; [symbol]
represents a single or a double bond along Y; and [symbol]
represents the alpha or beta anomer depending on the position of R₁;
a stereoisomer, salt, hydrate, solvate, or pharmaceutically acceptable crystal of the compound of Formula I;
a compound of Formula II: 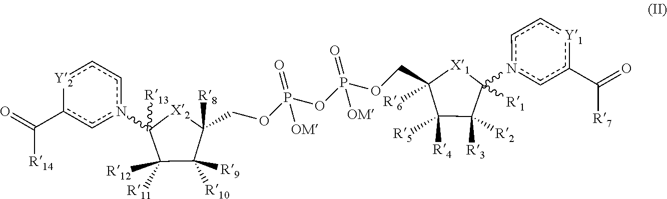
wherein
X′₁ and X′₂ are each independently selected from among, CH₂, S, Se, CHF, CF₂, and C═CH₂;
R′₁ and R′₁₃ are each independently selected from among H, azido, cyano, a C1-C8 alkyl, a C1-C8 thio-alkyl, a C1-C8 heteroalkyl, and OR, wherein R is selected from H and a C1-C8 alkyl;
R′₂, R′₃, R′₄, R′₅, R′₉, R′₁₀, R′₁₁, R′₁₂ are each independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;
R′₆ and R′₈ are each independently selected from among H, an azido, a cyano, a C₁-C₈ alkyl and OR, wherein R is selected from H and a C₁-C₈ alkyl;
R′₇ and R′₁₄ are each independently selected from among H, OR, NHR, NRR′, NH—NHR, SH, CN, N₃ and halogen; wherein R and R′ are independently selected from H and un (C₁-C₈) alkyl aryl;
Y′₁ and Y′₂ are each independently selected from among CH, CH₂, C(CH₃)₂, or CCH₃;
M′ is selected from H or a suitable counter ion; [symbol]
represents a single or double bond depending on Y′₁ and Y′₂; and [symbol]
represents an alpha or beta anomer depending on the position of R′₁ and R′₁₃;
a stereoisomer, salt, hydrate, solvate, or pharmaceutically acceptable crystal of the compound of Formula II; or
a combination thereof.

2. The method of claim 1, comprising:
administering to the subject a therapeutically effective amount of nicotinamide riboside or dihydronicotinamide riboside.

3. The method of claim 1, further comprising:
administering the NMN, the pharmaceutically acceptable derivative thereof, or the pharmaceutically acceptable salt thereof to the subject in a therapeutically effective amount to reduce allodynia associated with visceral nociceptive pain.

4. The method of claim 1, further comprising:
administering the NMN, the pharmaceutically acceptable derivative thereof, or the pharmaceutically acceptable salt thereof to the subject in a therapeutically effective amount to reduce hyperalgesia associated with visceral nociceptive pain.

5. The method of claim 1, wherein the pain is pain caused by a urinary tract infection.

6. The method of claim 1, wherein the NMN, the pharmaceutically acceptable derivative thereof, or the pharmaceutically acceptable salt thereof is administered to the subject in combination with at least one other therapeutic agent.

7. The method of claim 6, wherein the at least one other therapeutic agent is selected from among antibiotics, antifungals, antivirals, and combinations thereof.

8. The method of claim 6, wherein the at least one other therapeutic agent is an analgesic.

9. The method of claim 1 further comprising:
administering to the subject a composition comprising:
the NMN, the pharmaceutically acceptable derivative thereof, or the pharmaceutically acceptable salt thereof, and
at least one pharmaceutically acceptable excipient.

10. The method of claim 9, wherein the composition further comprises at least one other therapeutic agent.

11. The method of claim 1, wherein the pain is a pain in a urogenital (or genitourinary) system.

12. The method of claim 1, comprising:
administering to the subject the compound of Formula I, wherein X is oxygen; or
administering to the subject the compound of Formula II, wherein X′₁ and X′₂ each independently represent oxygen.

13. The method of claim 1, comprising:
administering to the subject the compound of Formula I, wherein R₈ is NH₂; or
administering to the subject the compound of Formula II, wherein X′₁ and X′₂ each independently represent NH₂.

14. The method of claim 1, comprising:
administering to the subject the compound of Formula I, wherein R₂, R₃, R₄, and R₅ are each independently hydrogen or OH; or
administering to the subject the compound of Formula II, wherein R′₂, R′₃, R′₄, R′₅, R′₉, R₁₀, R′₁₁, R′₁₂ are each independently hydrogen or OH.

15. The method of claim 1, comprising:
administering to the subject the compound of Formula I, wherein R₁ and R₆ each independently represent hydrogen; or
administering to the subject the compound of Formula II, wherein R′₁, R′₆, R′₈, and R′₁₃ each independently represent hydrogen.

16. The method of claim 1, comprising:
administering to the subject the compound of Formula I, wherein Y represents OH or CH₂; or
administering to the subject the compound of Formula II, wherein Y′₁ and Y′₂ each independently represent OH or CH₂.

17. The method of claim 1, comprising:
administering to the subject the compound of Formula I, wherein R₇ is P(O)(OH)₂.

18. The method of claim 1, comprising:
administering to the subject the compound of Formula I, wherein the compound of Formula I is selected from the group consisting of:

| Compound: | Structure: |
|---|---|
| I-C (beta) | 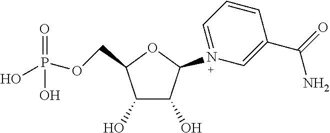 |
| I-D (alpha) | 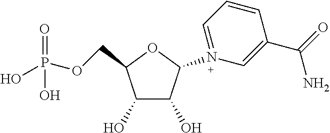 |
| I-G (beta) | 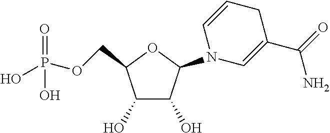 [SMILES: NC(=O)C1=CN([C@@H]2O[C@H](COP(=O)(O)O)[C@@H](O)[C@H]2O)C=CC1] |
| I-H (alpha) | 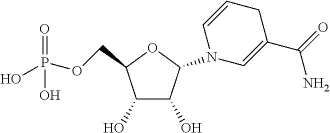 [SMILES: NC(=O)C1=CN([C@H]2O[C@H](COP(=O)(O)O)[C@@H](O)[C@H]2O)C=CC1] |
| Chem.7 (beta) | 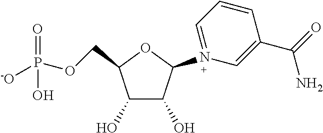 |
| Chem.3 (alpha) | 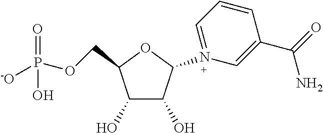 |

and salts, hydrates, solvates, and pharmaceutically acceptable crystals thereof.

19. The method of claim 1, comprising:
administering to the subject the compound of Formula II, wherein the compound of Formula II is selected from the group consisting of:

| Compound | Structure: |
|---|---|
| II-A (beta, beta) | 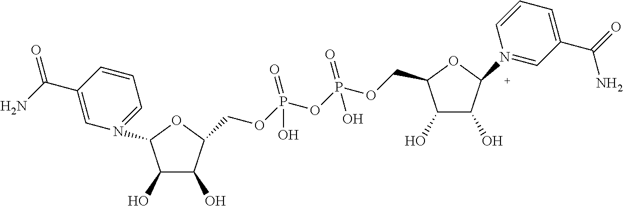 |
| II-B (beta, alpha) | 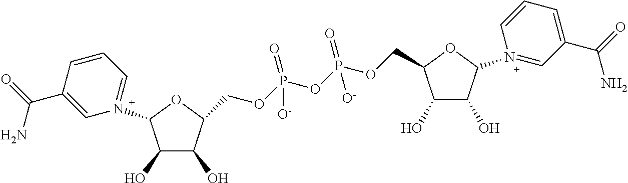 |
| II-C (alpha, alpha) | 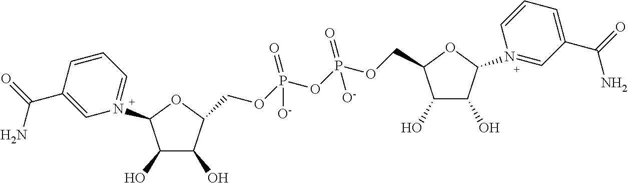 |
| II-D (beta, beta) | 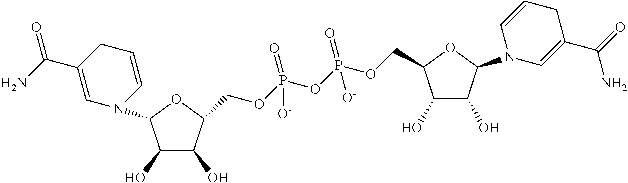 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| II-E (beta, alpha) | 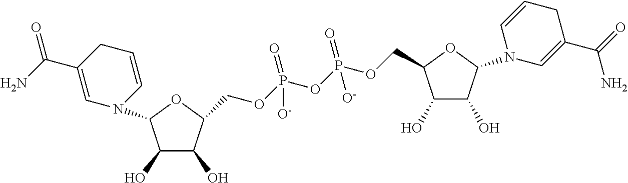 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| II-F (alpha, alpha) | 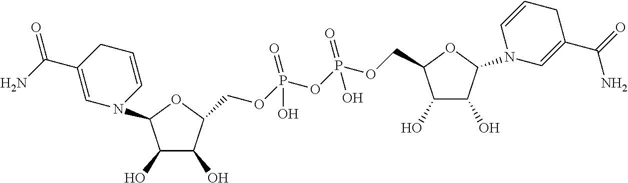 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

and salts, hydrates, solvates, and pharmaceutically acceptable crystals thereof.

20. The method of claim 1, wherein the NMN, the pharmaceutically acceptable derivative thereof, or the pharmaceutically acceptable salt thereof is administered to the subject orally or parenterally.
